(19) United States
(12) Patent Application Publication
(10) Pub. No.: US 2014/0051939 A1
(43) Pub. Date: Feb. 20, 2014

(54) OBTAINING PHYSIOLOGICAL MEASUREMENTS USING EAR-LOCATED SENSORS

(71) Applicant: Robert G. Messerschmidt, Los Altos, CA (US)

(72) Inventor: Robert G. Messerschmidt, Los Altos, CA (US)

(73) Assignee: Rare Light, Inc., Mountain View, CA (US)

(21) Appl. No.: 13/588,884

(22) Filed: Aug. 17, 2012

(51) Int. Cl. A61B 5/0205; A61B 5/0215; A61B 5/053; A61B 5/01; A61B 5/042; A61B 5/103; A61B 5/00; A61B 5/024

(52) U.S. Cl. 600/301; 600/300; 600/485; 600/549; 600/379; 600/587; 600/483; 600/547

(57) ABSTRACT

An apparatus and method for obtaining one or more physiological measurements associated with a user using ear-located sensors is disclosed herein. One or more of different types of sensors are configured to engage a user's ear. In some cases, the sensors will be included in one or both of a pair of earphones to capture physiological parameters. A portable device is configured to be in communication with the earphones to receive physiological parameters from the sensor(s) therein, and to potentially provide control signals to the sensors or other components in the earphones. The portable device determines physiological measurements corresponding to the received physiological parameters. The portable device is also configured to provide a user interface to interact with the user regarding the physiological measurements.

### CROSS-REFERENCE TO RELATED APPLICATIONS

This application is related to U.S. patent application Ser. No. ______ entitled “Obtaining Physiological Measurement Using Ear-Located Sensors” (Attorney Docket No. 3256.011US1) filed concurrently herewith.

### TECHNICAL FIELD

The present disclosure relates to obtaining physiological measurements of a user through use of ear-located sensors, and in particular embodiments, to obtaining physiological measurements through use of a portable device coupled to the sensors, which in some embodiments will be located within earphones.

### BACKGROUND

The current standard of care for blood pressure measurement is using a brachial cuff in the doctor's office or at home. Brachial cuff measurements include oscillometric measurements in which an air inflated cuff is positioned radially around a patient's arm in the vicinity of his/her brachial artery. Using a brachial cuff, however, is cumbersome and inadequate for a number of reasons. The cuff is uncomfortable and may even cause bruising. Brachial cuff measurements are susceptible to motion artifacts. Air pressure cuff devices tend to be large and not amendable to miniaturization. Brachial cuff measurements are also inadequate for thoroughly understanding a patient's blood pressure and changes in blood pressure. High blood pressure can be missed at the doctor's office if a patient's blood pressure is only high at certain times of the day. In this case, the opportunity to diagnose and treat high blood pressure is missed. Conversely, the patient may exhibit high blood pressure only when at the doctor's office. In this case, the patient may be unnecessarily placed on daily medication to lower blood pressure. Moreover, brachial cuff measurements provide peripheral blood pressure measurements (e.g., blood pressure at the arteries in the arms or legs) which can differ from central blood pressures (e.g., blood pressure at or near the aorta). For diagnostic and treatment purposes, central blood pressure measurements are preferred because they are a more accurate indicator of cardiovascular health.

Increasingly, the standard of care is moving toward ambulatory, non-invasive methods of obtaining physiological measurements. In the case of blood pressure measurements, a plurality of measurements obtained over a 24 hour or longer time period are of increasing importance in the practice of medicine. Such measurements provide better diagnosis and/or treatment of cardiovascular problems. Blood pressure is an important health statistic for overall health and wellness. When miniaturizing or configuring blood pressure measuring devices for home use, increasing their accuracy is an important consideration. Especially since patients are less well-versed in how to take measurements than medical personnel, it would be beneficial for measurement accuracy to be more or less built into the measurement device.

Other types of physiological measurements that may be tracked by individuals over an extended period of time and which are of value for overall health and wellness include, but are not limited to, electrocardiogram (ECG), body fat, and body water content measurements. So that individuals need not carry around multiple devices, it would be beneficial if a single device could capture one or more types of physiological measurements. It would also be beneficial if individuals can use an already existing device, which they would carry around anyway, to perform physiological measurement functions.

### BRIEF SUMMARY

As described herein, one or more sensor assemblies configured to engage a user by insertion in the ear can used in combination with any of various configurations of portable devices (such as, for example, a smart phone, a tablet, etc.) to obtain a variety of physiological measurements associated with the user. One or more of different types of sensors are included in one or more sensor assemblies to capture physiological parameters. In many embodiments, the sensor assemblies will be in the form of earphones, capable of also communicating audio information to a user ears. A portable device is configured to be in communication with the sensor assemblies to receive physiological parameters, and in some cases provide control signals to the sensor assemblies. In some embodiments, the physiological parameters are sensed in the absence of any applied stimulation to the user, and thus the parameters are monitored “passively.” The portable device determines physiological measurements corresponding to the received physiological parameters. The portable device is also configured to provide a user interface to interact with the user regarding the physiological measurements. The processing and communication capabilities of the portable device can be harnessed to provide a beginning-to-end measurement experience to the user. Physiological measurements include, but are not limited to, central aortic blood pressure measurements, carotid/central blood pressure measurements, ECG measurements, core body temperature measurements, skin surface temperature measurements, stress level indications, galvanic skin response measurements, body water content measurements, and/or body fat content measurements.

### BRIEF DESCRIPTION OF THE DRAWINGS

Some embodiments are illustrated by way of example and not limitations in the figures of the accompanying drawings, in which:

FIG. 1 illustrates an example system for obtaining one or more types of physiological measurements according to some embodiments.

FIGS. 2A-2D illustrates example portable devices used to obtain physiological measurements according to some embodiments.

FIGS. 3A-3C illustrates an example flow diagram for obtaining one or more physiological measurements using the system of FIG. 1 according to some embodiments.

FIG. 4 illustrates an example block diagram showing modules configured to facilitate the process of the flow diagram of FIGS. 3A-3C according to some embodiments.

FIG. 5 illustrates example user interface screens at the portable device providing sensor positioning instructions and measurement selection options according to some embodiments.

FIGS. 6A-6M illustrates example sensor configurations for the system of FIG. 1 according to some embodiments.

FIG. 7 illustrates an example configuration of the right and left earphones that includes a plurality of sets of sensors according to some embodiments.

FIG. 8 depicts a block diagram representation of an example architecture for the controller assembly according to some embodiments.

The headings provided herein are for convenience only and do not necessarily affect the scope or meaning of the terms used.

### DETAILED DESCRIPTION

The following detailed description refers to the accompanying drawings that depict various details of examples selected to show how the present invention may be practiced. The discussion addresses various examples of the inventive subject matter at least partially in reference to these drawings, and describes the depicted embodiments in sufficient detail to enable those skilled in the art to practice the invention. Many other embodiments may be utilized for practicing the inventive subject matter than the illustrative examples discussed herein, and many structural and operational changes in addition to the alternatives specifically discussed herein may be made without departing from the scope of the inventive subject matter.

In this description, references to “one embodiment” or “an embodiment,” or to “one example” or “an example” mean that the feature being referred to is, or may be, included in at least one embodiment or example of the invention. Separate references to “an embodiment” or “one embodiment” or to “one example” or “an example” in this description are not intended to necessarily refer to the same embodiment or example; however, neither are such embodiments mutually exclusive, unless so stated or as will be readily apparent to those of ordinary skill in the art having the benefit of this disclosure. Thus, the present invention can include a variety of combinations and/or integrations of the embodiments and examples described herein, as well as further embodiments and examples as defined within the scope of all claims based on this disclosure, as well as all legal equivalents of such claims.

For the purposes of this specification, a “processor-based system” or “processing system” as used herein, includes a system using one or more microprocessors, microcontrollers and/or digital signal processors or other devices having the capability of running a “program,” (all such devices being referred to herein as a “processor”). A “program” is any set of executable machine code instructions, and as used herein, includes user-level applications as well as system-directed applications or daemons.

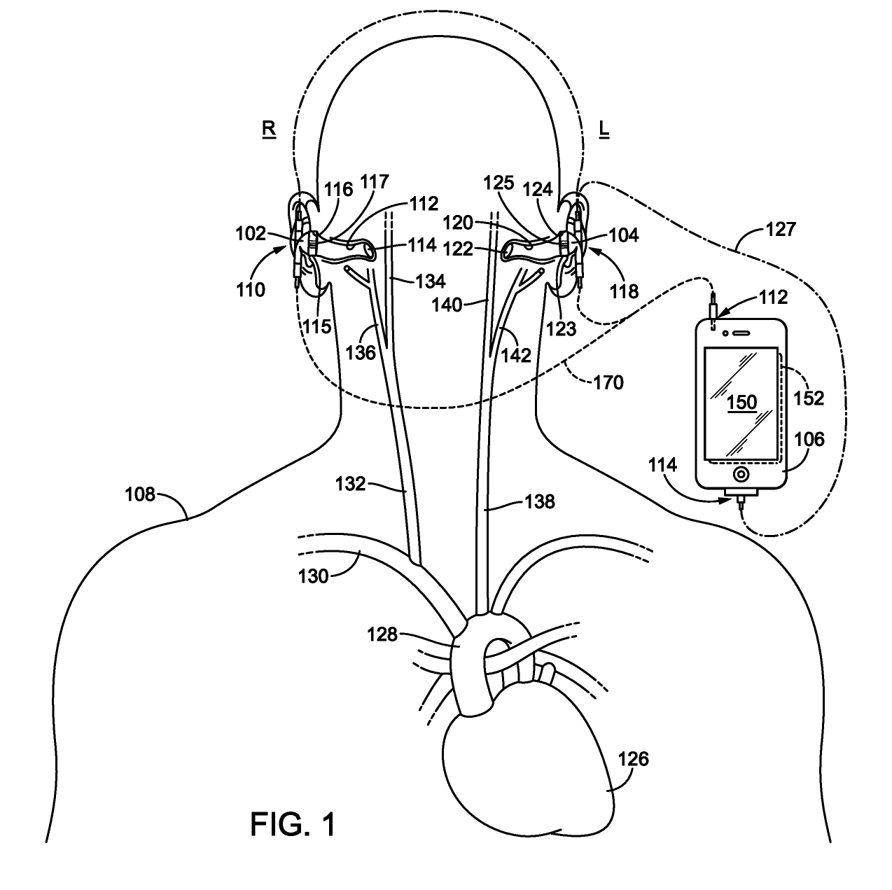

FIG. 1 illustrates an example system 100 for obtaining one or more types of physiological measurements through use of example embodiments of sensor assemblies. In the described examples, the sensor assemblies are all in the form of one or more earphones that are capable of communicating other audio communication from an attached portable device. The system 100 includes a right earphone 102, a left earphone 104, and a portable device 106. The right and left earphones 102, 104 are shown inserted into the right and left ears 110, 118, respectively, of a user 108. The right earphone 102 is shown inserted into a right ear canal 112. The right earphone 102 is configured to form a sealed chamber or cavity within the right ear 110, wherein the sealed chamber is bounded by the (inner) perimeter of the right ear canal 112, a tympanic membrane 114, and the right earphone 102. The right earphone 102 is configured to form a (compression) seal along a perimeter 116 at or near the entry of the right ear canal 112. The right earphone 102 has a plurality of sides, the outer perimeter/circumference of one side 115 forming a (sufficient) seal with the inner perimeter/circumference 116 of the right ear canal 112. The right earphone 102 also has two opposing sides, in which one of the opposing sides (a side 117) is the side closest to the tympanic membrane 114 and a boundary of the sealed cavity formed within the right ear 110.

The left earphone 104 is shown inserted into a left ear canal 120. The left earphone 104 is configured to form a sealed chamber within the left ear 118, wherein the sealed chamber is bounded by the (inner) perimeter of the left ear canal 120, a tympanic membrane 122, and the left earphone 104. The left earphone 104 is configured to form a seal along a perimeter 124 at or near the entry of the left ear canal 120. The left earphone 104 has a plurality of sides, the outer perimeter/circumference of one side 123 forming a (sufficient) seal with the inner perimeter/circumference 124 of the left ear canal 120. The left earphone 104 also has two opposing sides, in which one of the opposing sides (a side 125) is the side closest to the tympanic membrane 122 and a boundary of the sealed cavity formed within the left ear 118.

The right and left earphones 102, 104 can be, for example, noise-reduction earphones that work by acoustically isolating the ear canals 112, 120 from the outside world, by sealing against the ear canals. As described later herein, such isolation has the effect of forming a sealed chamber in the ear canal that will facilitate different forms of acoustic and/or pressure measurements. The right and left earphones 102, 104 include one or more sensors (not shown in FIG. 1) to detect one or more physiological parameters, from which physiological measurements are calculated. Details regarding the sensors included in the right and left earphones 102, 104 are discussed in detail below.

The right and left earphones 102, 104 (also referred to as right and left ear buds) include an audio line 170 for a wired connection to the portable device 106. In some embodiments, the audio line 170 plugs into an earphone jack 112 included on the portable device 106. The audio line 170 will typically include multiple conductors, at least over a portion of its length, and can be sued to provide audio signals to the right and left earphones 102, 104, for typical earphone operations. Additionally, as will be apparent from the discussion to follow, audio line 170 may also be configured to send signals and/or power to sensors in right and left earphones 102, 104, and to receive data from such sensors. The right and left earphones 102 may be connected to a portable device 106 through any of multiple configurations of connections, depending in part of the configuration of ports on the portable device 106. In one example embodiment, speakers in right and left earphones 102, 104 can be coupled through audio line 170 to earphone jack 112; while sensors in either (or both) headphones may be coupled through a separate a sensor line 127 to a separate port on the portable device 106. In this configuration, sensor line 127 can plug into, for example, a 30-pin connector or a universal serial bus (USB) port included on the portable device 106. In this embodiment, the sensor line 127 will provide power (in some instances) and uni- or bi-directional communication between the right and left earphones 102, 104 and the portable device 106, as described in detail below.

As an alternative, all connections between the portable device 106 and right and left earphones 102, 104 could be through a separate port from the earphone jack 112, such as the above identified 30-pin or USB connector. As yet another alternative, if the headphone jack of the portable device (which, again, may include an external module coupled to a phone, tablet or other portable device), may have a earphone jack configured to provide both conventional audio functionality through earphones 102, 104, as well as other power and/or data communication as necessary to provide the physiological monitoring functions described herein. Alternatively, in some embodiments, the audio line 170 and/or the sensor line 127 may be omitted, and the right and left earphones 102, 104 and or the sensors therein may wirelessly communicate with the portable device 106, such as through a Bluetooth connection. In this case, to the extent that a power source is necessary, at least one power source may be included in the right and left earphones 102, 104.

A portion of the user's 108 cardiovascular system is shown in FIG. 1. In particular, a heart 126 is shown located slightly on the left side of the user's 108 body. From the heart 126 is an aortic arch 128, and from the aortic arch 128 there exists a subclavian artery 130 on the right side of the user's 108 body. From the subclavian artery 130 exists a common carotid artery 132, which in turn splits into an internal carotid artery 134 and an external carotid artery 136 in the neck. The external carotid artery 136 is located near the right ear canal 112, less than approximately 25 mm from the right ear canal 112. On the left side of the user's 108 body, a common carotid artery 138 stems from the aortic arch 128. The common carotid artery 138 splits into an internal carotid artery 140 and an external carotid artery 142. The external carotid artery 142 is located near the left ear canal 120, less than approximately 25 mm from the left ear canal 120. Blood pumped by the heart 126 travels from the aortic arch 128, to each of the subclavian artery 130 and common carotid artery 138, and then it travels to each of the internal and external carotid arteries 134, 136 from the common carotid artery 138. Similarly, blood located in the subclavian artery 130 travels to the common carotid artery 132, and from there to each of the internal and external carotid arteries 134, 136. Because the arterial path on the left side of the body has a slightly shorter path to the heart than the right side (e.g., left side of the body does not have a subclavian artery between the aortic arch 128 and the common carotid artery 138), blood pumped by the heart 126 at a given time arrives first at the external carotid artery 142 (on the left side).

The example portable device 106 of FIG. 1 includes a touch sensor panel 150 (also referred to as a touch screen) and a controller assembly 152. The touch sensor panel 150 includes an array of pixels to sense touch event(s) from a user's finger, or other body part, or a stylus or similar object. Examples of touch sensor panel 150 include, but are not limited to, capacitive touch sensor panels, resistive touch sensor panels, infrared touch sensor panels, etc. The controller assembly 152 is configured to provide processing and control capabilities for the portable device 106. The controller assembly 152 can include machine-executable instructions stored in a machine-readable storage media, software applications (apps), circuitry and other hardware, and combinations of the above.

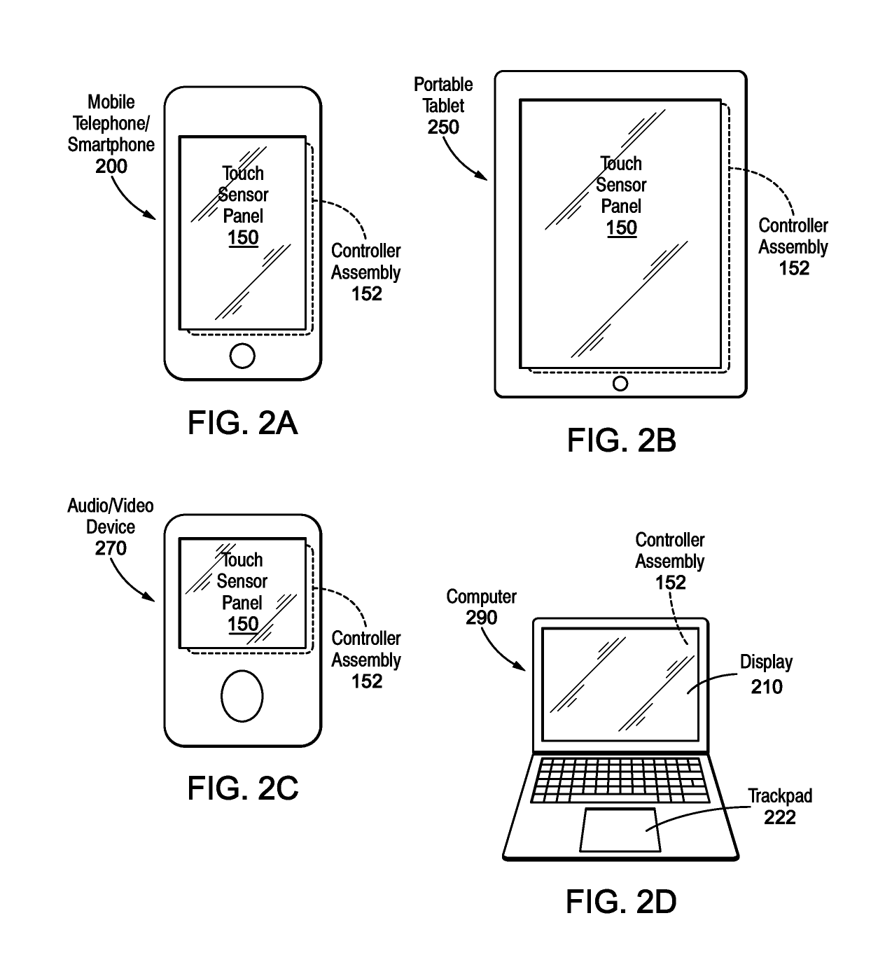

FIGS. 2A-2D illustrate examples of the portable device 106 according to some embodiments. A portable device includes any of a variety of processor-based devices that are easily portable to a user, including, for example, a mobile telephone or smart phone 200, a portable tablet 250, an audio/video device 270 (such as an iPod or similar multimedia playback device), a computer 290 such as a laptop or netbook, or a dedicated portable device specific for the purpose of making measurements of the types generally described herein; and further includes an external component that operatively couples to another portable device, such as through a USB port, a 30-pin port or another external interface port. Such external component can be in any of a variety of form factors, including a dongle coupled directly or through a cable to the port or another configuration that mechanically engages coupled portable device (such as a case structure, for example). Where one portable device is coupled to another portable device to function together, though each is a discrete “portable device,” the combination of the two devices should also be considered to be a “portable device” for purposes of this disclosure. As discussed in detail later herein, the portable devices will be used in combination with sensor assemblies configured to engage a user's ear; and in many embodiments the sensor assemblies can be in the form of earphones configured to communicate audio information to a user, and also adapted as described herein to sense physiological parameters of the user.

While many of the portable devices will be expected to include a touch screen, such is not necessarily required (see for example, computer 290 having a display, but not a touch screen), except for configurations herein which depend specifically on receiving inputs through such a touch screen, as will be apparent from the discussion to follow; though most embodiments will include some form of display though which to communicate with a user. Each of the portable devices includes a controller assembly 152 including one or more processors, which will provide the functionality of the device. Each portable device may also include additional controls or other components, such as: a power button, a menu button, a home button, a volume button, a camera, a light flash source for the camera, and/or other components to operate or interface with the device. In FIG. 2, the example touch screens 150 and controller assemblies 152 have been numbered similarly, though as will be readily apparent to those skilled in the art, such numbering is not intended to suggest that such structures will be identical to one another, but merely that the identified elements generally correspond to one another.

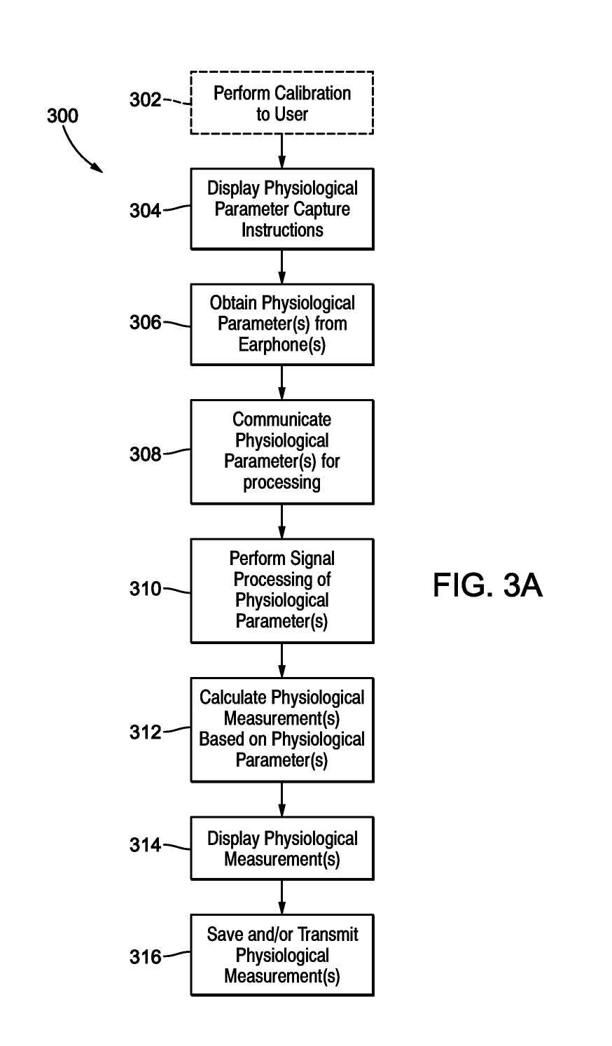

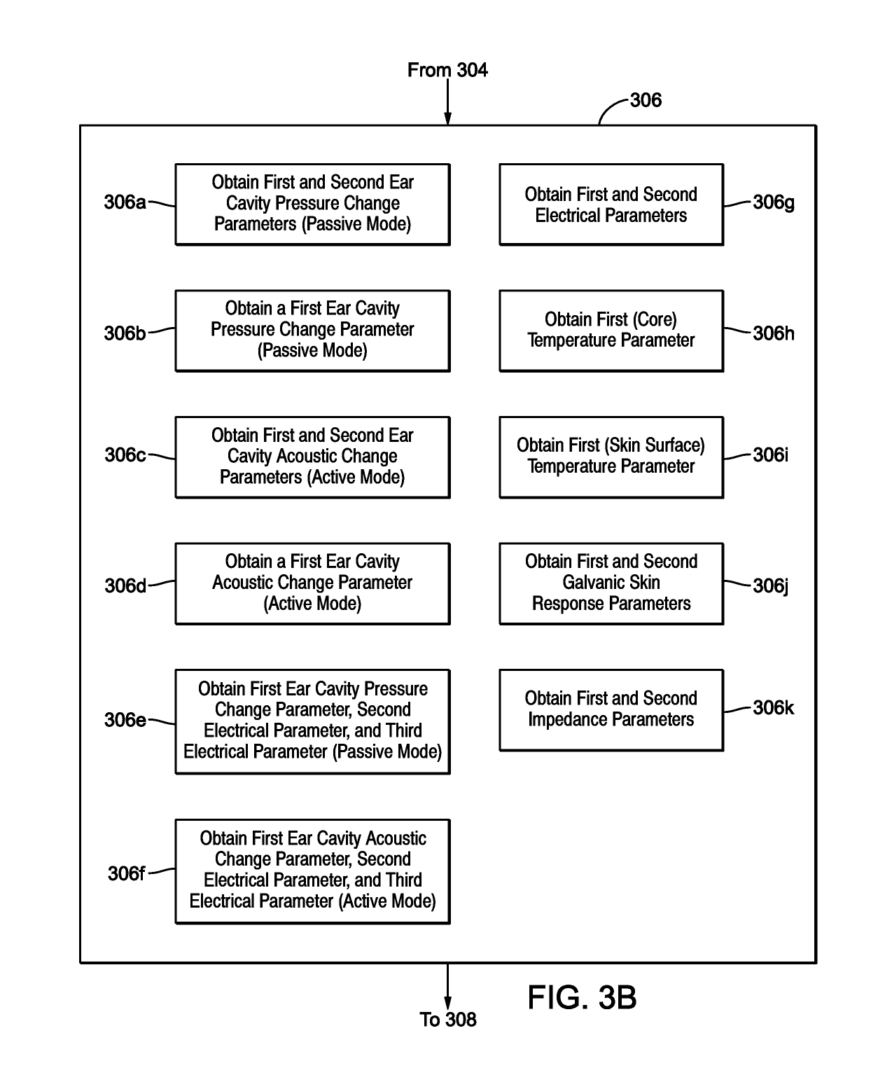

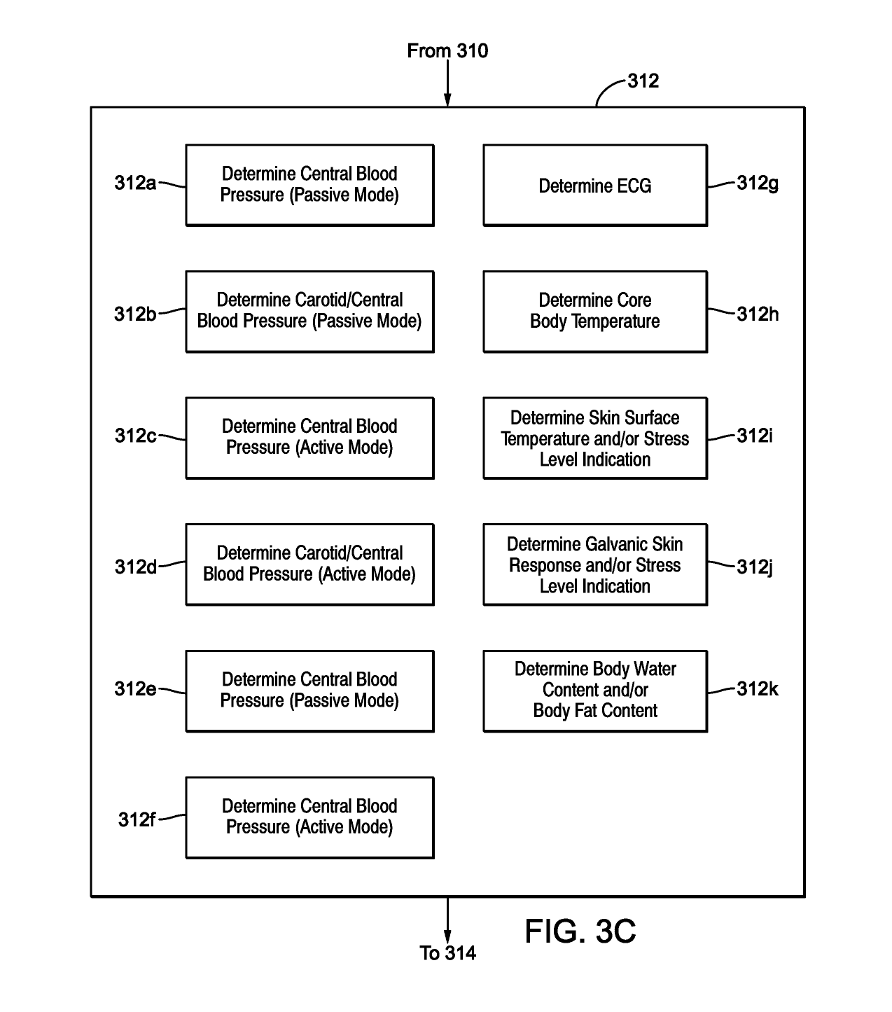

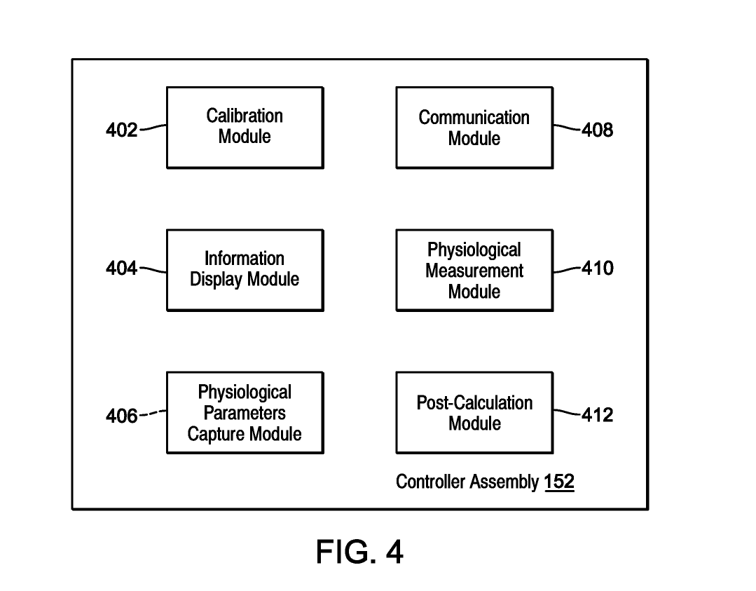

FIGS. 3A-3C illustrate an example flow diagram 300 for obtaining one or more physiological measurements using the system 100. FIG. 4 illustrates an example embodiment block diagram showing modules configured to facilitate the process of flow diagram 300. FIGS. 3B-C and 4 reference taking measurements in a passive mode and/or in an active mode (each of which will be discussed in more detail, later herein). Embodiments can be based upon passive measurement systems only, active measurement systems only, or combinations of elements and measurement methodologies of each type of system. Accordingly, modules for taking both types of measurements are depicted in FIG. 4; and steps of taking both types of measurement are depicted in FIG. 3B-C. As will be apparent to those skilled in the art, the modules included in a system, and the steps performed to take measurements will be a function of the types of measurements selected to be taken, and the type of system selected to perform the measuring. And thus various embodiments will be expected to diverge from the examples of FIGS. 3 and 4, depending on the selections made for such systems.

In most systems, the modules shown in FIG. 4 are included in the controller assembly 152 of the portable device 106. However, in other systems, one or more of the modules (or just some portion of the structure and/or functionality thereof) can also be located in an external component electrically and/or communicatively coupled (and often physically coupled) to another portable device (as described earlier herein), and in some such embodiments one or more processors in such external component will execute some or all software-implemented functionality in such module, and/or hardware in in such dongle or accessory device can execute other functionality of the module.

The modules of FIG. 4 include conceptual modules representing instructions encoded in a computer readable storage device. When the instructions encoded in the computer readable storage device are executed by the controller assembly 152 it causes the performing of certain tasks as described in the example herein. In this example, both the computer readable storage device and the processing hardware/firmware to execute the encoded instructions stored in the storage device are components of the portable device 106. Though, as noted above, some or all of the instructions may be stored in a computer readable device in external component in electrical and/or data communication with the portable device. Although the modules shown in FIG. 4 are shown as distinct modules, it should be understood that they can be implemented as fewer or more modules than in the depicted example. It should also be understood that any of the modules may communicate with one or more components external to the portable device 106 via a wired or wireless connection. FIGS. 3A-3C will be described in conjunction with FIG. 4.

At block 302, a calibration module 402 is configured to perform calibration with respect to the user 108 in preparation of obtaining usable physiological measurement(s). The need to perform calibration depends on the type of physiological measurement to be obtained. In one embodiment, calibration is performed for measurements that use blood pulse transit time or blood pulse velocity. An information display module 404 may be configured to cause the portable device 106 to display calibration instructions on the touch sensor panel 150. For example, the calibration instructions may instruct the user 108 to use a brachial cuff to obtain one or more blood pressure measurements while simultaneously having the right and left earphones 102, 104 properly inserted in his/her ears. The brachial cuff blood pressure measurement(s) may be automatically transmitted to the portable device 106, or alternatively the portable device 106 may provide input fields on the touch sensor panel 150 for the user 108 to manually input the blood pressure obtained from the brachial cuff. At or approximately the same time that the brachial cuff measurement(s) is being made, the portable device 106 is configured to obtain one or more blood pressure measurements using the right and/or left earphones 102, 104. Using both sets of blood pressure measurements, the calibration module 402 is configured to determine one or more scaling factors to properly calibrate the conversion of the blood pulse transit time (or blood pulse velocity) obtained using the right and/or left earphones 102, 104 from the user 108 to a central (e.g., aortic) blood pressure measurement. The conversion function between the blood pulse transit time (or blood pulse velocity) and desired blood pressure measurement is known, as discussed in detail below, but the scaling up or down of the conversion function for each particular user is obtained from the calibration process.

In another embodiment, calibration is performed for physiological measurements using skin impedance detection (e.g., body fat content measurement). The information display module 404 may be configured to cause display of calibration instructions relating to skin impedance measurements on the touch sensor panel 150. Calibration instructions may instruct the user 108 to enter his/her height, weight, age, and gender prior to measuring the user's skin impedance. The calibration module 402 is configured to use the user-specific information to calibrate the user's skin impedance measurement to report an accurate body fat content information to the user.

The type of calibration(s) may be automatically determined based on the types of sensor(s) included in the right and left earphones 102, 104. Alternatively, the calibration(s) are performed based on the types of physiological measurements specified by the user 108. One or more calibrations may be performed at the block 302 for a particular user. Calibration(s) may be performed each time before a physiological measurement is made, or alternatively may be performed periodically (e.g., once a month), or in some cases may be a one-time event for a given user. The calibration schedule for one type of physiological measurement may be different for different types of physiological measurements.

In still another embodiment, the calibration block 302 may be omitted. For example, in the case of electrocardiogram (ECG) measurements, in some applications, no calibration with respect to particular individuals is required to calculate an ECG measurement from electro-physiological parameters detected from individuals. As another example, no calibration may be required for providing body temperature measurements to users.

Next at block 304, the information display module 404 is configured to cause display of physiological parameter(s) capture instructions on the touch sensor panel 150. The physiological parameters capture instructions include one or more user interface screens providing instructions, tips, selection options, and other information to the user 108 to facilitate proper detection of physiological parameter(s) corresponding to desired physiological measurement(s). In one embodiment, the user 108 is instructed to insert the right and left earphones 102, 104 into the respective right and left ears 110, 118, and to ensure a seal with the respective ear canals. The right and left earphones 102, 104 may include a sensor to confirm that an adequate seal has been formed in each of the right and left ear canals 112, 120. In some embodiments, since the portable device 106 can either “know” or automatically detect the sensor types included in the right and left earphones 102, 104, the user 108 may not be required to specify what physiological measurement(s) are desired. Proper positioning of the right and left earphones 102, 104 with respect to the user 108 can be sufficient to start obtaining physiological parameter(s) pertaining to the user 108 at block 306.

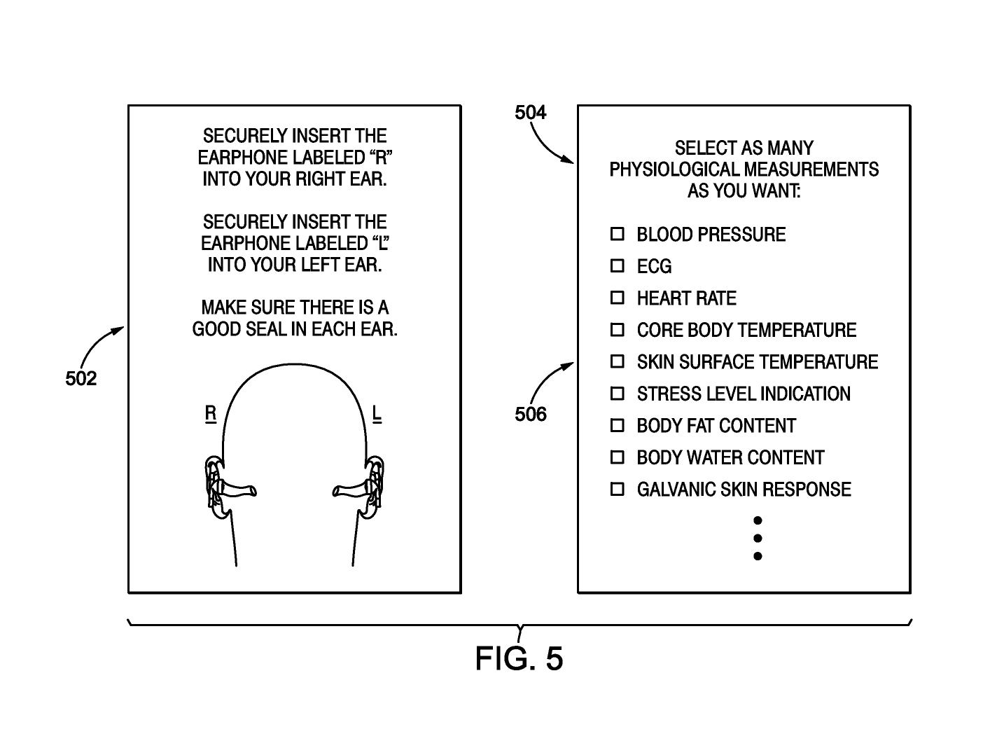

In another embodiment, the user 108 is instructed to insert the right and left earphones 102, 104 into the respective right and left ears 110, 118, and to ensure a seal with the respective ear canals. The right and left earphones 102, 104 can include a sensor to confirm that an adequate seal has been formed in each of the right and left ear canals 112, 120, or the adequacy of a seal can be determined from the sensor measurements themselves. Next, the portable device 106 may present a list of physiological measurements that may be obtained from the right and left earphones 102, 104, and request the user 108 to select one or more desired physiological measurements from the presented list. FIG. 5 illustrates an example user interface screen 502 at the portable device 106 providing sensor positioning instructions, and an example user interface screen 504 at the portable device 106 providing measurement selection options 506 according to some embodiments. When the user 108 makes selection(s) from among the measurement selection options 506, the portable device 106 is configured to obtain physiological parameters corresponding to the user selection(s) at the block 306.

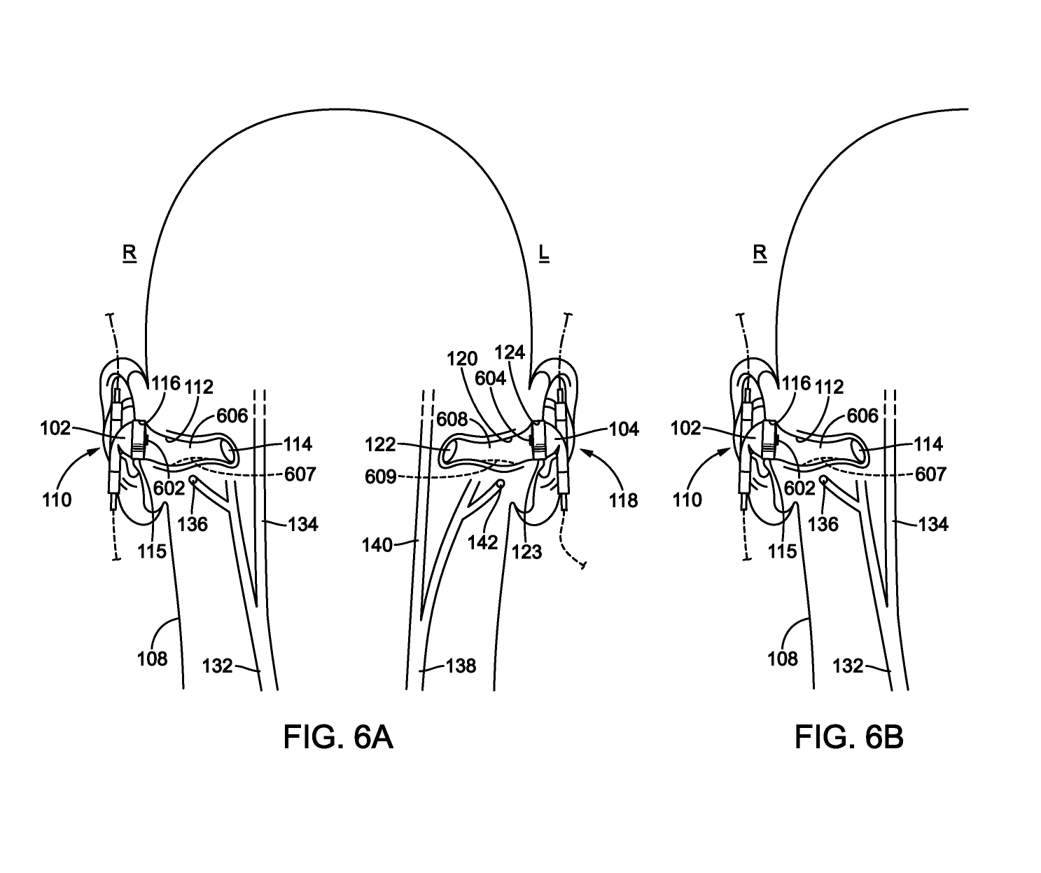

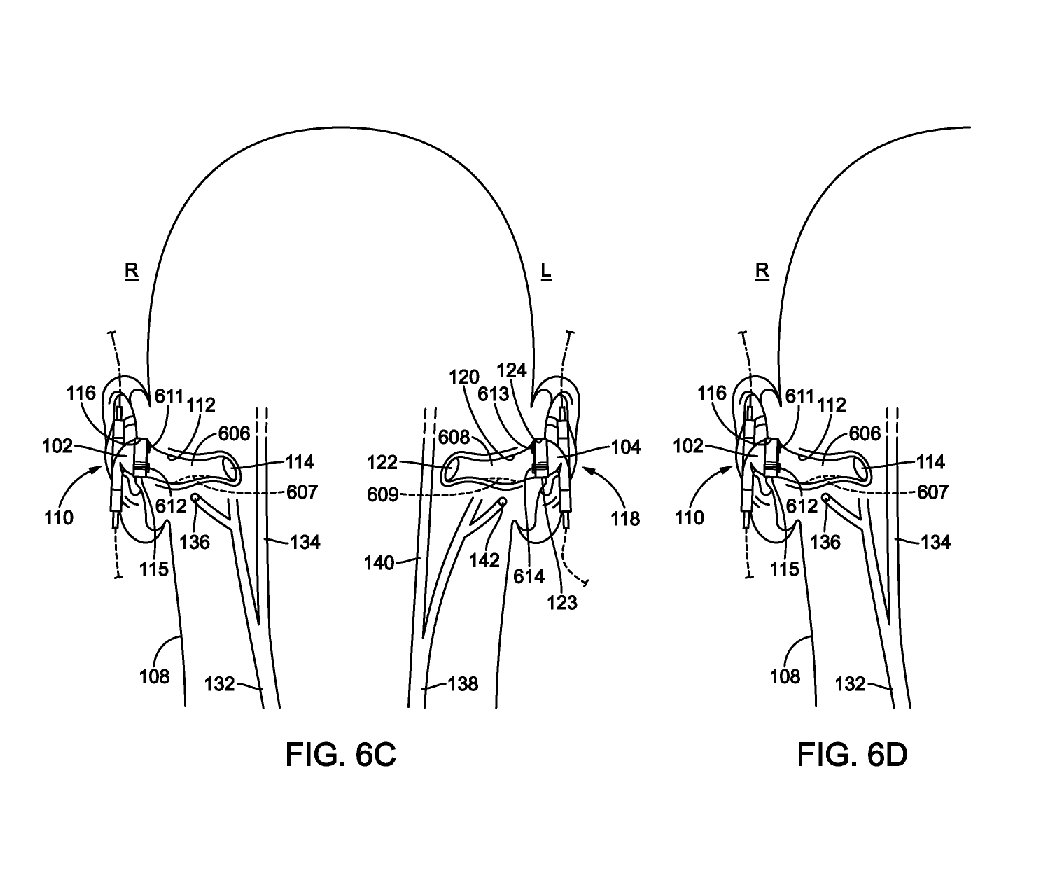

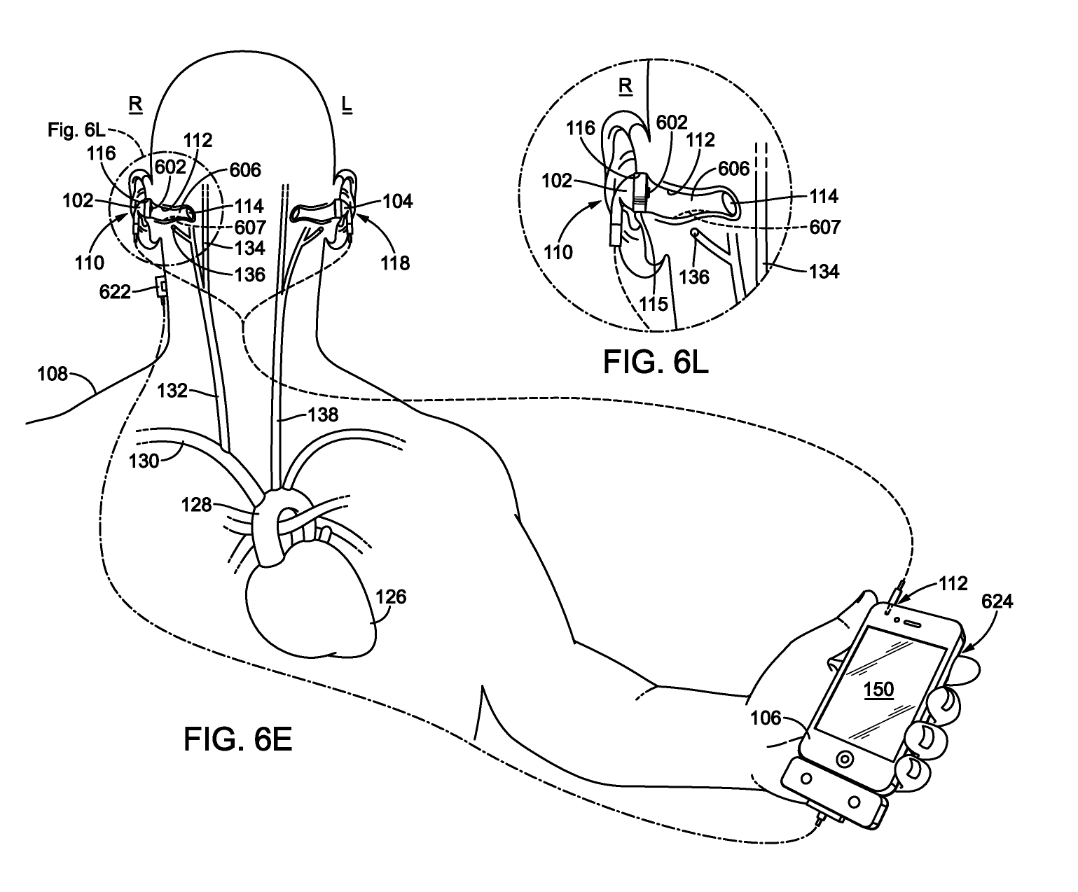

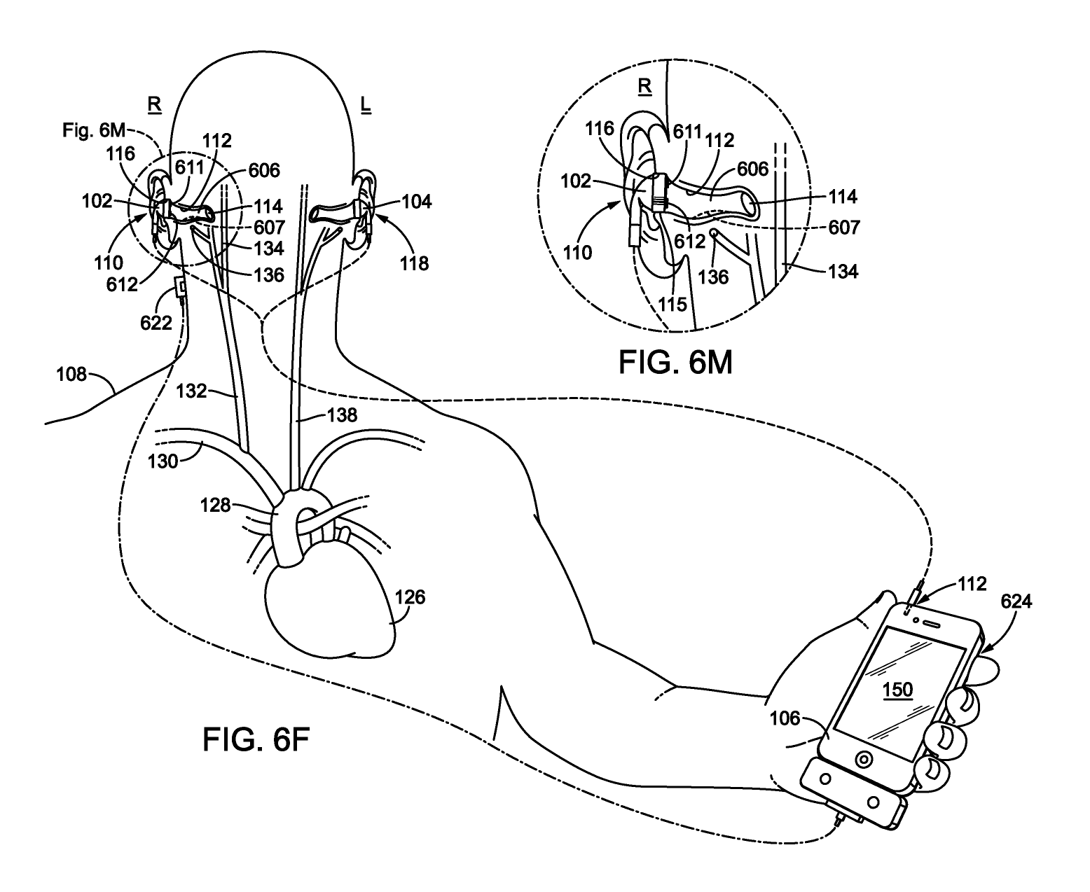

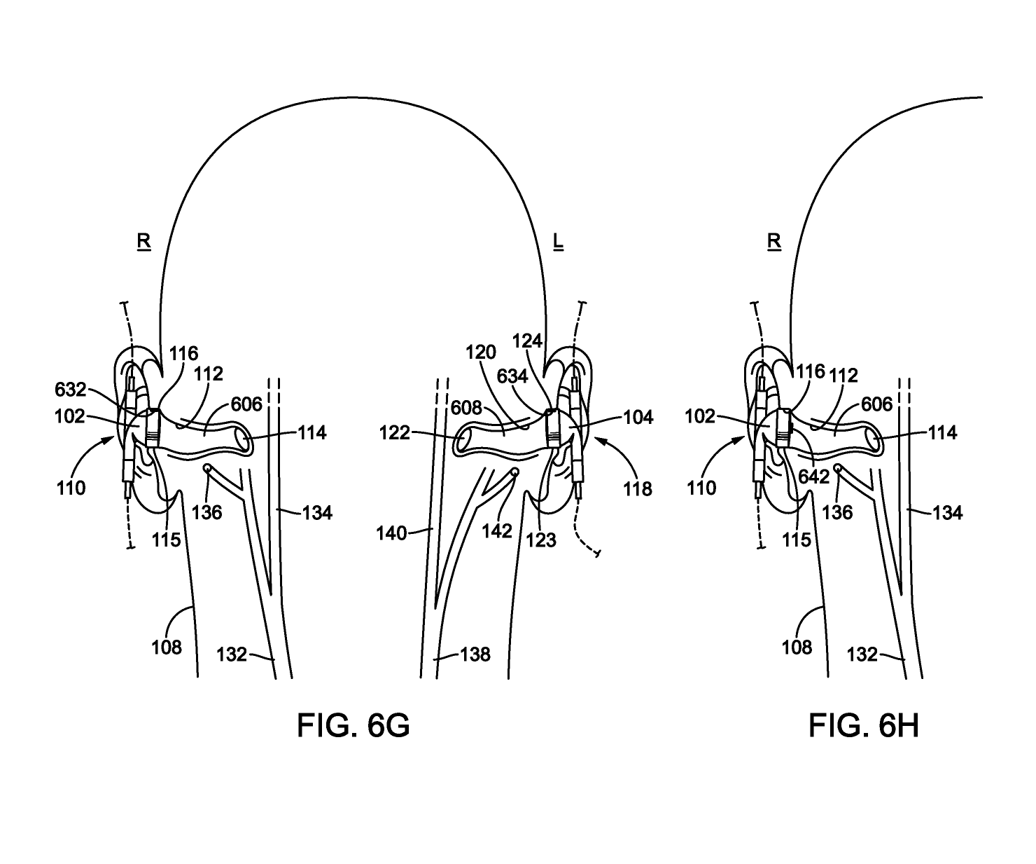

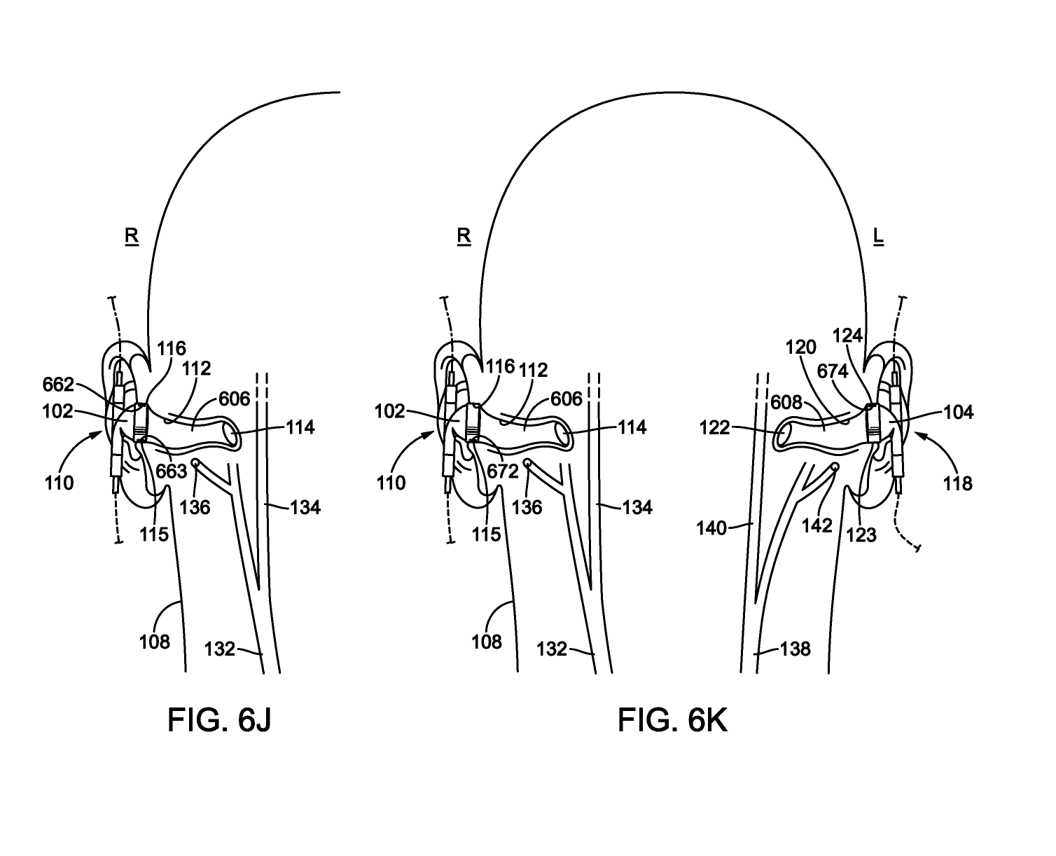

Next at the block 306, a physiological parameters capture module 406 is configured to control the sensor(s) included in the right and/or left earphones 102, 104 corresponding to the physiological measurements designated (implicitly or explicitly) in the block 304, to cause those sensor(s) to obtain physiological parameter(s) from the user 108. The physiological parameters capture module 406 provides the necessary input, timing, and/or power signals to these sensors for periodic or essentially continuous data capture. The sensors can be powered from a power line included in the audio line 170, a dedicated power line included in the sensor line 127, or a power source included in the right and left earphones 102, 104 in the case of wireless operation. As discussed with respect to FIGS. 3B and 6A-6M, one or more sensors are included in the right and/or left earphones 102, 104 to detect particular physiological parameters from the user 108. A variety of sensor configurations are implemented to obtain a blood pressure measurement, an ECG measurement, a core body temperature measurement, a skin surface temperature measurement, a stress level measurement (e.g., galvanic skin response), body water content measurement (e.g., skin impedance), and/or body fat content measurement (e.g., skin impedance). In FIGS. 6A-6M, one or more lines or leads between the right and left earphones 102, 104 and/or between the right and left earphones 102, 104 and the portable device 106 are not shown for ease of illustration.

FIG. 3B illustrates example sub-blocks 306a-k of the block 306 according to some embodiments. At sub-block 306a, the physiological parameters capture module 406 is configured to obtain a first ear cavity pressure change parameter from the right earphone 102 and a second ear cavity pressure change parameter from the left earphone 104. As shown in FIG. 6A, the right earphone 102 includes a pressure sensor such as, for example, a pressure transducer 602 provided along a side of the right earphone 102 that forms a bound of a sealed ear cavity 606 formed by sealing the right ear canal 112. The pressure transducer 602 is configured to obtain the first ear cavity pressure change parameter. The left earphone 104 includes a pressure transducer 604 provide along a side of the left earphone 104 that forms a bound of a sealed ear cavity 608 formed by sealing the left ear canal 120. The pressure transducer 604 is configured to obtain the second ear cavity pressure change parameter. Each of the pressure transducer sensors 602, 604 includes a piezo pressure transducer configured to detect pressure change, though any other form of pressure sensor suitable for the measurements described herein may be utilized.

With each pumping of the blood by the heart 126, a blood bolus travels through the arteries, including the external carotid arteries 136 and 142, providing a traveling blood pulse. With the external carotid artery 136 located close to the (right) sealed ear cavity 608, each blood pulse within the external carotid artery 136 causes at least a portion of the bounds of the sealed ear cavity 608 to deflect inward (e.g., depicted deflection 607). This deflection represents a slight decrease in the volume of the sealed ear cavity 608, and this in turn results in a slight pressure increase in the sealed ear cavity 608. The pressure transducer 602 included in the right earphone 102 senses this pressure change (as a function of time). Thus, each pressure change detection corresponds to the presence of a blood pulse in the portion of the external carotid artery 136 located proximate to the sealed ear cavity 606. The pressure transducer 604 similarly obtains pressure change measurements for the left ear 118—each blood pulse arriving at the external carotid artery 142 causing a deflection 609 of the bounds of the sealed ear cavity 608.

When more than one pressure change measurement is made by a given pressure transducer, a train of blood pulses or a blood pulse waveform (each waveform peak indicative of a blood pulse) is obtained as a function of time. The pressure transducer sensors 602, 604 provide voltage outputs corresponding to the right and left carotid circulatory waveforms, respectively, as a function of time. There is a slight difference in the time arrival of a blood pulse associated with a given blood bolus between the right and left ears 110, 118, in which the blood pulse arrives first at the left ear 118 because the external carotid artery 142 on the left side of the body has a shorter path to the heart 126. As discussed below with respect to block 312, this difference in the pulse arrival time (PAT) between the right and left ears 110, 118 relates to a pulse wave velocity (PWV), and the pulse wave velocity, in turn, correlates to a central (e.g., aortic) blood pressure measurement. The right and left blood pulse waveforms are provided to the portable device 106 for conversion to a central (e.g., aortic) blood pressure measurement.

The sensor configuration shown in FIG. 6A includes a passive mode of measuring the central blood pressure (e.g., the sensor output is not a response to an intentionally introduced input to the sealed ear cavity) that is not susceptible to motion artifact. Once the right and left earphones 102, 104 are normally positioned within the right and left ears 110, 118 (e.g., each is positioned to serve its regular function of noise-reduction by forming a seal with the boundaries forming the ear canal), the earphones are also automatically properly positioned to capture the pulse arrival times. Moreover, by including such pressure transducers in the right and left earphones 102, 104, the arterial length distance between the pressure transducer sensors 602, 604 is fixed—unlike with traditional measurement methods—and for an adult, this distance never changes.

At sub-block 306b, the physiological parameters capture module 406 is configured to obtain a first ear cavity pressure change parameter from one of the right or left earphones 102, 104. The first ear cavity pressure change parameter is obtained using a pressure transducer included in an earphone, as discussed above for FIG. 6A. FIG. 6B shows this single earphone sensor configuration with respect to the right earphone 102, in which like numbers correspond to like numbers in FIG. 6A. However, it should be understood that the pressure transducer can instead be located in the left earphone 104. Alternatively, a pressure transducer can be located in each of the right and left earphones 102, 104 as shown in FIG. 6A, and the first ear cavity pressure change parameter may be obtained from just one of the right or left earphones 102, 104.

The first ear cavity pressure change parameter is converted by the portable device 106 to a carotid arterial blood pressure measurement. In this case, it is assumed that the carotid arterial blood pressure is sufficiently identical to the central aortal blood pressure that the two can be considered equivalent. For this reason, a second ear cavity pressure change parameter from the other earphone is not required to calculate the difference in the pulse arrival time (which relates to PWV, which in turn relates to central aortal blood pressure).

At sub-block 306c, the physiological parameters capture module 406 is configured perform an active mode measurement, by obtaining a first ear cavity acoustic change parameter from the right earphone 102 and a second ear cavity acoustic change parameter from the left earphone 104 in response to an introduced input. As shown in FIG. 6C, the right earphone 102 includes a speaker 611 (as typically included for normal earphone operations) and a microphone 612, both provided along a side of the right earphone 102 that forms a bound of the sealed ear cavity 606 formed by sealing the right ear canal 112. The left earphone 104 also includes a speaker 613 as typically included for normal earphone operations, and a microphone 614, both provided along a side of the left earphone 104 that forms a bound of the sealed ear cavity 608 formed by sealing the left ear canal 120. The microphones 612, 614 can be of any of a variety of configurations, including, e.g., tonometers, acoustic sensors, or indirect pressure sensors, etc. The first and second ear cavity acoustic change parameters may also referred to as ear cavity indirect pressure change parameters.

A known acoustic input is provided by each of the right and left earphones 102, 104 (via speakers 611, 613) to the sealed ear cavities 606, 608, respectively. In response, the microphones 612, 614 detect the sound emitted from the speakers 611, 613, respectively, as well as the sound reflected from the walls of the sealed ear cavities 606, 608, respectively. When a blood pulse passes through the portion of the external carotid artery 136 that is nearest the sealed ear cavity 606, the shape of the sealed ear cavity 606 deforms (e.g., deflection 607) and becomes more rigid. These changes to the shape and rigidity of the sealed ear cavity 606 causes the profile of the sound reflected from the walls of the sealed ear cavity 606 to change. For example, the amplitude of the reflected wave may be reduced and/or the phase delay of the reflected wave may increase due to changes in the sealed ear cavity 606 induced by a blood pulse. The microphone 612 detects such acoustic change (as a function of time). The microphone 614 similarly detects the acoustic change (as a function of time) caused by blood pulses traveling through the portion of the external carotid artery 142 that is nearest the sealed ear cavity 608. The acoustic input provided by the speakers 611, 613 can be any type of sound, such as music, spoken words, a sound tone, within the human audible frequency range, outside the human audible frequency range, or any other acoustic waveform that can be emitted by the speakers 611, 613. In one embodiment, the acoustic input can be provided simultaneous with whatever sound (e.g., music) the user 108 is normally listening. In another embodiment, the acoustic input can be provided by itself, and in some instances, it may be at a frequency (or frequencies) inaudible to the user 108 to minimize intrusive sounds being provided to the user 108.

The first and second physiological parameters obtained at sub-block 306a are detection of pressure changes induced by the blood pulses. In the sensor configuration of FIG. 6C, the first and second physiological parameters are detection of acoustic changes induced by the blood pulses. These acoustic changes relate to the pressure change of the sealed ear cavities 606, 608. The pressure change, in turn, relates to the difference in the pulse arrival time between the right and left ears 110, 118 (similar to the discussion above for sub-block 306a). The difference in the pulse arrival time relates to the pulse wave velocity, and the pulse wave velocity, in turn, correlates to the central aortic blood pressure measurement. The right and left waveforms of the first and second ear cavity acoustic change parameters are provided to the portable device 106 for conversion to the central (e.g., aortic) blood pressure measurement.

The sensor configuration of FIG. 6C (also referred to as a tonometry method) facilitates active modes for measuring the central blood pressure. Similar to the passive mode described above, direct measure of tonometry changes to determine the central blood pressure is not susceptible to motion artifact. Once the right and left earphones 102, 104 are normally positioned within the right and left ears 110, 118 as described above, the earphones are also automatically properly positioned to capture the pulse arrival times. Also, as noted above, by including such microphones in the right and left earphones 102, 104, the arterial length distance between the microphones 612, 614 is fixed.

At sub-block 306d, the physiological parameters capture module 406 is configured to obtain a first ear cavity acoustic change parameter from one of the right or left earphones 102, 104. The first ear cavity acoustic change parameter is obtained using a microphone sensor included in an earphone, as discussed above for FIG. 6C. FIG. 6D shows this single earphone sensor configuration with respect to the right earphone 102, in which like numbers correspond to like numbers in FIG. 6C. However, as discussed previously, the microphone sensor can instead be located in the left earphone 104. Alternatively, a microphone can be located in each of the right and left earphones 102, 104 as shown in FIG. 6C, and the first ear cavity acoustic change parameter may be obtained from both or just one of the right or left earphones 102, 104.

The first ear cavity acoustic change parameter is converted by the portable device 106 to a carotid arterial blood pressure measurement. Again, it is assumed that the carotid arterial blood pressure is effectively identical to the central aortal blood pressure, enabling use of a measurement in a single ear.

At sub-block 306e, the physiological parameters capture module 406 is configured to obtain a passive mode measurement of first ear cavity pressure change parameter from one of the right or left earphones 102, 104 (e.g., via the pressure transducer 602), a second electrical parameter associated with a portion of the user's 108 artery closer to the heart 126 than the external carotid artery 136 (e.g., via a first electrode 622 in FIG. 6E), and a third electrical parameter from a portion of the user's body on the opposite side to the side where the second electrical parameter is obtained (e.g., via a second electrode 624 in FIG. 6E). Alternatively, the sensor placement to obtain the second and third electrical parameters (e.g., the placement of first and second electrodes 622, 624) can be anywhere across the midline—on opposite wrists, left wrist and a right finger, etc.

FIG. 6E shows a single earphone sensor associated with the right earphone 102 (the pressure transducer 602), in which like numbers correspond to like numbers in FIGS. 6A, 6B (depicted in greater detail in the enlargement of FIG. 6L). The first electrode 622 is placed on the right side of the user's 108 body and proximate a portion of the user's artery that is closer to the heart 126 than the external carotid artery 136. For example, the first electrode 622 can be placed on the right side of the neck, near the common carotid artery 132. The second electrode 624 may be provided on the portable device 106, such as the back or side of the portable device 106. When the user 108 naturally grips the portable device 106, for example, electrical contact is made between the second electrode 624 and the user's hand. When the first electrode 622 is placed on the right side of the user's body, the second electrode 624 should contact a portion of the left side of the user's body (e.g., user's left hand/finger). Although the above discussion is made with respect to the pressure sensor located in the right earphone 102 and the first electrode 622 placed on the right side of the user's body, it should be understood that the pressure sensor can instead be located in the left earphone 104, the first electrode 622 can be placed on the left side of the user's body (e.g., left side of the user's neck near the common carotid artery 138), and the second electrode 624 is contacted by a portion of the right side of the user's body (e.g., user's right hand/finger).

The pressure transducer 602 is configured to provide voltage outputs corresponding to the blood pulse waveform at the portion of the external carotid artery 136 nearest the sealed ear cavity 606 (in the same manner as discussed above with respect to sub-blocks 306a, b). The first and second electrodes 622, 624 are configured to detect electrical signals corresponding to ECG spikes. When the user 108 simultaneously makes contact with the first and second electrodes 622, 624, a complete electrical circuit is created including the user 108. The first and second electrodes 622, 624 permit the portable device 106 to capture electrical characteristics of the user 108, typically in the form of resistance measurements. The first and second electrodes 622, 624 detect an electrical signal corresponding to the depolarization of the heart 126 when blood is ejected from the heart 126. This electrical signal is referred to as an ECG spike. The given blood bolus ejected from the heart 126 travels through the arteries away from the heart 126 and at some later point in time reaches the external carotid artery 136 by the right ear 110. The arrival of the bolus at the external carotid artery 136 is detected by the pressure transducer 602. Because a given depolarization of the heart 126 occurs prior to the corresponding blood bolus arriving at the external carotid artery 136, the capture of the electrical signal corresponding to that heart depolarization occurs prior to the corresponding blood bolus arriving at the external carotid artery 136 (e.g., at the common carotid artery 132).

The detected blood pulse information as a function of time is provided to the portable device 106 for determining a central blood pressure measurement. Using the ECG spike timing information from the first and second electrodes 622, 624 and the corresponding blood pulse timing information from the pressure transducer 602 (e.g., blood pulse timing information from two locations) provides a method to determine the pulse wave velocity, which may be correlated to the central blood pressure.

Each of the first and second electrodes 622, 624 includes a conductive material such as a metallic material or another material having a sufficiently low electrical resistivity to allow function as an electrode for purposes of the intended measurements (e.g., for example: conductive hydrogel, conductive foam or runnerized material, silicon, conductive yarns including silver coated nylon, stainless steel yarn, silver coated copper filaments, silver/silver chloride, etc.). At least with respect to the second electrode 624, this electrode can include some portion (or the entirety) of various structural components associated with the portable device, such as: (1) the back of the portable device 106, (2) a side of the portable device 106, (3) an antenna of the portable device 106, (4) a button on the portable device 106, (5) a detachable external component (as discussed earlier herein) attached to the portable device 106, (6) the touch sensor panel 150 of the portable device 106, or (7) a sleeve or case encasing the portable device 106.

At sub-block 306f, the physiological parameters capture module 406 is configured to perform an active mode measurement of several parameters: a first ear cavity acoustic change parameter from one of the right or left earphones 102, 104 (e.g., via the microphone 612); a second electrical parameter associated with a portion of the user's 108 artery closer to the heart 126 than the external carotid artery 136 (e.g., via the first electrode 622 in FIG. 6F); and a third electrical parameter from a portion of the user's body on the opposite side to the side where the second electrical parameter is obtained (e.g., via the second electrode 624 in FIG. 6F). Alternatively, the sensor placement to obtain the second and third electrical parameters (e.g., the placement of first and second electrodes 622, 624) can be anywhere across the midline—on opposite wrists, left wrist and a right finger, etc.

As illustrated in FIGS. 6F and 6M, this sensor configuration is same as shown in FIG. 6E except that the microphone 612 and the speaker 611 are used to obtain the blood pulse timing information in the active mode instead of in the passive mode using the pressure transducer 602. Reference is made to the discussions above for sub-blocks 306c, d regarding the active mode using the microphone 612 and speaker 611. Reference is also made to the discussion above for sub-block 306e regarding use of blood pulse timing information obtained at one location in combination with ECG spike information obtained at another location.

Although FIG. 6F shows use of the speaker 611 and microphone 612 located in the right earphone 102 and the first electrode 622 placed on the right side of the user's body, as previously discussed relative to other embodiments, the speaker and microphone can instead be located in the left earphone 104, the first electrode 622 can be placed on the left side of the user's body (e.g., left side of the user's neck near the common carotid artery 138), and the second electrode 624 is contacted by a portion of the right side of the user's body (e.g., user's right hand/finger).

At sub-block 306g, the physiological parameters capture module 406 is configured to simultaneously obtain a first electrical parameter from the right earphone 102 and a second electrical parameter from the left earphone 104. An electrical circuit is completed by a first electrode 632 provided on the right earphone 102, a second electrode 634 provided on the left earphone 104 (see FIG. 6G), and the user 108. The first electrode 632 makes electrical contact with a portion of the right ear canal 112, such as a portion of the perimeter 116 at or near the entry of the right ear canal 112 where a seal is formed. The second electrode 634 makes electrical contact with a portion of the left ear canal 120, such as a portion of the perimeter 124 at or near the entry of the left ear canal 120 where a seal is formed.

The first and second electrodes 632, 634 obtain resistive measurements from one side of the user's body to the other side, which are provided to the portable device 106 for conversion into ECG measurements. Each of the first and second electrodes 632, 634 again include a conductive material as discussed above.

At sub-block 306h, the physiological parameters capture module 406 is configured to obtain a first temperature parameter from one of the right or left earphones 102, 104. FIG. 6H shows obtaining the first temperature parameter using a temperature sensor 642 included in the right earphone 102. The temperature sensor 642 is positioned to measure the temperature within the sealed ear cavity 606. For example, the temperature sensor 642 can include an infrared (IR) radiation detector configured to measure the amount of IR radiation in the right ear canal 112. The output of the temperature sensor 642 is provided to the portable device 106, and the portable device 106 converts the parameter into a core body temperature of the user 108. As with previous embodiments, although FIG. 6H shows the temperature sensor included in the right earphone 102, such a temperature sensor can alternatively be included in the left earphone 104.

At sub-block 306i, the physiological parameters capture module 406 is configured to obtain a first temperature parameter from one of the right or left earphones 102, 104. FIG. 6I shows obtaining the first temperature parameter using a temperature sensor 652 included in the right earphone 102. The temperature sensor 652 is positioned along a side of the right earphone 102 that is in contact with the perimeter 116 of the right ear canal 112. The temperature sensor 652 can include any suitable sensor, such as a thermocouple, thermopile, or resistance temperature detector (RTD) sensor. The output of the temperature sensor 652 is provided to the portable device 106, and the portable device 106 is configured to convert the output into a skin surface temperature of the user 108. Skin (surface) temperature relates, among other things, to the user's stress level. Typically in a stressful situation, a person's peripheral circulation (including skin circulation) decreases, which causes the skin temperature to decrease. As another example, a person's core body temperature typically differs from his/her skin temperature. However, when there is a significant difference between the core body temperature and skin temperature, this is indicative of health issues, such as an adverse drug reaction. As with other sensors, although FIG. 6I shows the temperature sensor included in the right earphone 102, such a temperature sensor can alternatively be included in the left earphone 104.

At sub-block 306j, the physiological parameters capture module 406 is configured to obtain both a first impedance parameter and a second impedance parameter from one of the right or left earphones 102, 104. FIG. 6J illustrates a first electrode 662 configured to obtain the first impedance parameter and a second electrode 663 configured to obtain the second impedance parameter. The first electrode 662 is located along the side of the right earphone 102 such that it comes into electrical contact with a portion of the right ear canal 112, that forms the seal. The second electrode 663 is located along a side of the right earphone 102 opposite to that of the first electrode 662, such that it also comes into electrical contact with the opposing portion of the right ear canal 112, e.g., comes into contact with the opposing portion of the perimeter 116 that forms the seal. Each of the first and second electrodes 662, 663 is formed of a conductive material as discussed earlier herein in reference to FIG. 6E.

The first and second electrodes 662, 663 form an electrical circuit with the user 108. The first and second electrodes 662, 663 measure the moisture level of the user's skin at the contact areas, the moisture level indicative of a galvanic skin response. Galvanic skin response, in turn, is an indication of a person's stress level (or the opposite of stress, relaxation level). Although FIG. 6J shows the two electrodes included in the right earphone 102, such electrodes can alternatively be included in the left earphone 104.

At sub-block 306k, the physiological parameters capture module 406 is configured to obtain a first impedance parameter from the right earphone 102 and a second impedance parameter from the left earphone 104. As shown in FIG. 6K, the first impedance parameter is obtained by a first electrode 672 included in the right earphone 102. The first electrode 672 is located along the side of the right earphone 102 such that it comes into electrical contact with a portion of the right ear canal 112. The second impedance parameter is obtained by a second electrode 674 included in the left earphone 104. The second electrode 674 is located along the side of the left earphone 104 such that it comes into electrical contact with a portion of the left ear canal 120. Note that although FIG. 6K shows the first electrode 672 contacting the bottom of the right ear canal 112 and the second electrode 674 contacting the top of the left ear canal 120, any perimeter portion of the ear canal can be contacted (e.g., top, bottom, frontal side, back side, etc.) as long as the first and second electrodes 672, 674 respectively contact opposing sides of the user's body.

Each of the first and second electrodes 672, 674 is again formed of a conductive material as discussed earlier herein. The first and second electrodes 672, 674 operate on the circuit-completion concept to obtain impedance measurements between one side of the user's body to the other side. Such measurements are provided to the portable device 106 for conversion into body water content measurements and/or body fat content measurements.

Each of FIGS. 6A-6M illustrates a set of sensors (one, two, or three sensors) placed at specific locations on the user's 108 body, and which are configured to obtain a set of physiological parameters corresponding to a particular physiological measurement. For example, FIG. 6A shows using a pressure transducer located at each of the right and left earphones 102, 104 to obtain central aortic blood pressure measurements, while FIG. 6H shows using a single temperature sensor located at one of the right or left earphones 102, 104 to obtain core body temperature measurements. In one embodiment, the right and left earphones 102, 104 can be configured as shown in any of FIGS. 6A-6M to obtain one type of physiological measurement.

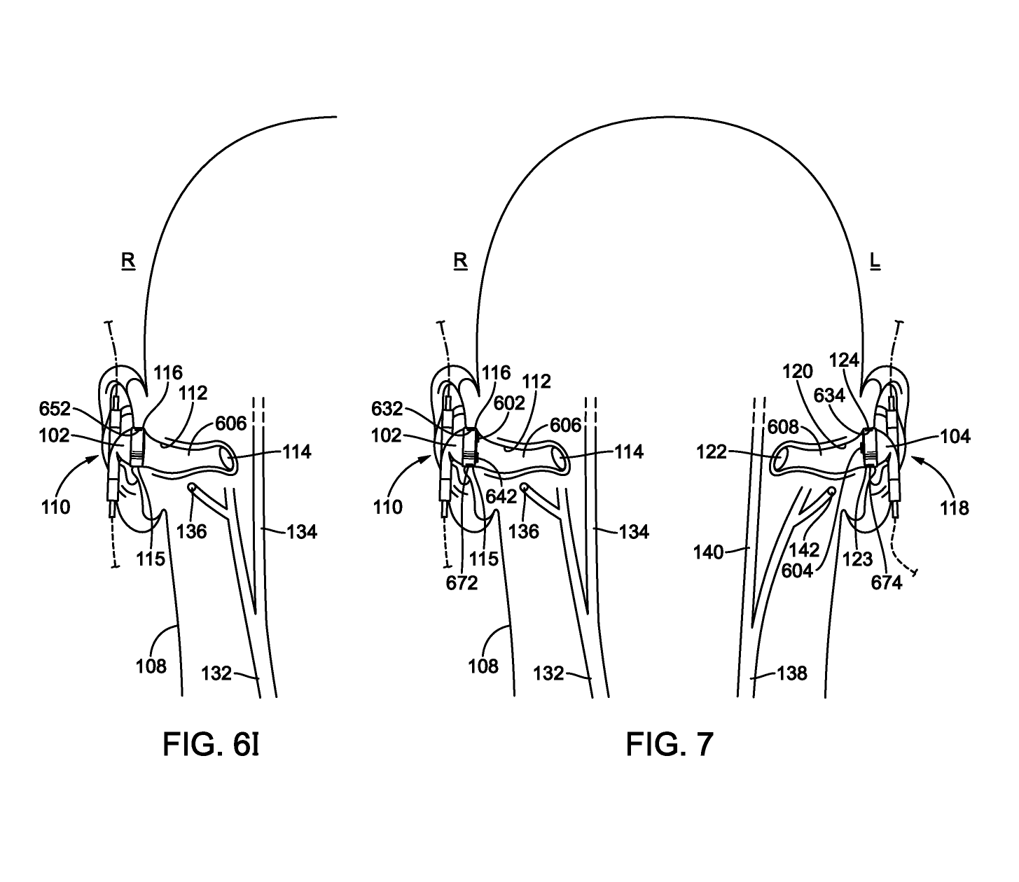

In another embodiment, the right and left earphones 102, 104 can be configured to include more than one set of sensors shown in each of FIGS. 6A-6M to obtain more than one type of physiological measurements, either sequentially or simultaneously. For instance, FIG. 7 illustrates an example configuration of the right and left earphones 102, 104 that includes a plurality of sets of sensors. The right and left earphones 102, 104 include: (1) pressure transducer sensors 602, 604 for central aortic blood pressure measurement, (2) first and second electrodes 632, 634 for ECG measurement (and/or heart rate measurement), (3) first temperature sensor 642 for core body temperature measurement, and (4) first and second electrodes 672, 674 for body water content and/or body fat content measurement. Note that in some embodiments, one of the set of first and second electrodes 632, 634 or the set of first and second electrodes 672, 674 can be operated in a manner to enable obtaining both of the ECG measurements (e.g., resistive type of measurement) and the body water content and/or body fat content measurements (e.g., impedance type of measurement). As such, in some embodiments one of the set of first and second electrodes may be omitted from FIG. 7 without lose of measurement capabilities.

Provided below are other example combinations of sets of sensors that can be included in a pair of sealing-types of earphones to form medical-use earphones. The possible subset(s) of each of the combinations listed below are not provided but it is contemplated that such subset(s) can also be implemented. Alternatively, any other combinations of sets of sensors may be included in pairs of sealing-type of earphones.

| | | | | | |
|---|---|---|---|---|---|
| FIG. 6A | FIG. 6B | FIG. 6C | FIG. 6D | FIG. 6E | FIG. 6F |
| FIG. 6G | FIG. 6G | FIG. 6G | FIG. 6G | FIG. 6G | FIG. 6G |
| FIG. 6H | FIG. 6H | FIG. 6H | FIG. 6H | FIG. 6H | FIG. 6H |
| FIG. 6I | FIG. 6I | FIG. 6I | FIG. 6I | FIG. 6I | FIG. 6I |
| FIG. 6J | FIG. 6J | FIG. 6J | FIG. 6J | FIG. 6J | FIG. 6J |
| FIG. 6K | FIG. 6K | FIG. 6K | FIG. 6K | FIG. 6K | FIG. 6K |

Returning to FIG. 3A, once one or more of the physiological parameter(s) have been obtained, such parameters are communicated by signals from the sensors to the portable device 106 for processing and conversion into the appropriate physiological measurement(s) (block 308). The physiological parameters are communicated via wire connections (e.g., sensor line 127) or wireless connections (e.g., Bluetooth). Depending on the frequency of the physiological parameters from a given set of sensors and/or the number of types of physiological parameters from different set of sensors, physiological parameters from a given set of sensors can be singularly provided to portable device 106 (e.g., in essentially real-time) or those parameters can be combined with physiological parameters from one or more other sets of sensors for combined transmission to the portable device 106. A communication module 408 is configured to coordinate communication of obtained physiological parameters from the right and left earphones 102, 104 to the portable device 106.

Next at block 310, a physiological measurement module 410 is configured to control signal processing and other pre-processing functions to ready the obtained physiological parameter signals suitable for conversion to appropriate physiological measurements. Depending on the state of the physiological parameters received at the portable device 106, one or more of the following processing functions may occur: analog-to-digital (A/D) conversion, demultiplexing, amplification, one or more filtering (each filter configured to remove a particular type of undesirable signal component such as, noise, known input component, etc.), other pre-conversion processing, and the like. The processing can be performed by hardware, firmware, and/or software. The type and extent of signal processing can vary depending on the type of physiological parameters. For example, physiological parameter signals obtained from the microphones 612, 614 may undergo digitization, filtering (to at least remove the introduced acoustic input), and other signal conditioning. Whereas physiological parameter signals obtained from the first and second electrodes 632, 634 may require little signal processing, e.g., potentially merely A/D conversion. Additionally, in some embodiments, some or all of the signal processing may be performed by the right and left earphones 102, 104. For example, if the raw output of a certain sensor requires signal processing unique to that sensor (e.g., unique circuitry) and/or the sensor packaging can easily include signal processing functionalities, the raw output of a sensor may be processed prior to transmission to the portable device 106. An advantage of this approach is that the portable device 106 requires less circuitry, for example, that is dedicated for one function. Another advantage is that the portable device 106 may receive uniformly processed physiological parameter signals from a variety of sensors.

Next at block 312, the physiological measurement module 410 is configured to determine appropriate physiological measurements from the (conditioned) physiological parameter signals. Block 312 includes additional processing to translate physiological parameters into physiological measurements that are well-understood by the user 108. FIG. 3C illustrates example sub-blocks 312a-k of the block 312 according to some embodiments. Like suffixes in sub-blocks 312a-k and sub-blocks 306a-k correspond with each other (e.g., sub-block 312a corresponds to sub-block 306a). Each of the sub-blocks 312a-k include use of a particular algorithmic method or functional relationship(s) established between given physiological parameters and physiological measurements to convert or translate those physiological parameters to appropriate physiological measurements.

At sub-block 312a, the physiological measurement module 410 is configured to determine a central (aortic) blood pressure measurement based on the first and second ear cavity pressure change parameters obtained from pressure transducer sensors 602, 604 shown in FIG. 6A. Each of the first and second ear cavity pressure change parameters includes a blood pulse waveform as a function of time. The slight difference in the arrival of each given blood pulse between the right ear 110 and the left ear 118, referred to as a difference in pulse arrival time (Δ PAT), is derived from the two blood pulse waveforms. The Δ PAT relates to the pulse wave velocity (PWV), and PWV relates to the central aortic blood pressure (also referred to as the central arterial blood pressure (CABP). In other words, Δ PAT enjoys a functional relationship with the central aortic blood pressure: Δ PAT=f(CABP).

In one embodiment, the translation or conversion of measured Δ PAT to CABP can be performed using known algorithmic methods that specify the quantitative relationship or correlation between Δ PAT and CABP. For example, reference is made to Garcia-Ortiz, L, et al., “Comparison of two measuring instruments, B-pro and SphygmoCor system as reference, to evaluate central systolic blood pressure and radial augmentation index,” Hypertension Research, 1-7 (2012) available at http://www.laalamedilla.org/evident/Publicaciones/article.pdf, which provides correlations between peripheral and central blood pressure measurements (see for example Table 2 of the article). The peripheral blood pressure measurements were obtained from actual PWV and distance measurements on test subjects.

In another embodiment, the functional relationship between Δ PAT and CABP can be empirically derived. For example, a human study can be conducted in which three simultaneous measurements are obtained from each subject: (1) Δ PAT by hooking up the subject to the right and left earphones 102, 104 including the pressure transducer sensors 602, 604, respectively, (2) a CABP by actually measuring the blood pressure at the subject's aorta during cardiac catheterization (adding a pressure sensor to a catheter that is snaked through the subject's arteries, including positioning the pressure sensor on the catheter in the aortic arch 128 to directly measure CABP), and (3) a brachial blood pressure (brachial BP) using a brachial cuff. A relatively small number of subjects are currently considered to be sufficient to establish basic correlation parameters, such as about 50 subjects. The three simultaneous measurements for a given subject provide an empirical relationship between Δ PAT, CABP, and brachial BP. The empirical relationships from all the subjects are averaged, resulting in an empirically-derived functional relationship between Δ PAT and CABP.

The empirically-derived relationship between Δ PAT, CABP, and brachial BP can also be used to calibrate each particular user from which Δ PAT will be obtained. In particular, as discussed above with respect to block 302, a Δ PAT measurement and a brachial BP measurement are simultaneously obtained from a given user during calibration. Using these two known measurements associated with the given user in comparison with the derived functional relationship between Δ PAT and brachial BP, a scaling factor applicable to the particular user can be determined, to adjust the CABP value up or down. Subsequently, when a Δ PAT measurement is actually obtained from that user (e.g., using the sensor configuration of FIG. 6A), the portable device 106 can convert the measured Δ PAT to a provisional brachial BP using the derived functional relationship between Δ PAT and brachial BP and additionally apply the (calibration) scaling factor applicable to that user to the provisional brachial BP to determine a final brachial BP. The final brachial BP, in turn, is converted into the CABP using the derived functional relationship between brachial BP and CABP.

At sub-block 312b, the physiological measurement module 410 is configured to determine a carotid/central blood pressure measurement based on the first ear cavity pressure change parameter obtained from one of the pressure transducer sensors 602, 604, as shown in FIG. 6B. The first ear cavity pressure change parameter includes a blood pulse waveform as a function of time at a portion of the external carotid artery. This blood pulse waveform is representative of the external carotid artery blood pressure (also referred to as the carotid blood pressure). The conversion to the carotid blood pressure can be performed using known conversion algorithmic methods, such as Principle components regression. Assuming the carotid blood pressure is sufficiently the same as CABP, a sensor configuration using a single pressure transducer provides the central and carotid blood pressure measurements.

At sub-block 312c, the physiological measurement module 410 is configured to determine a central (aortic) blood pressure measurement based on the first and second ear cavity acoustic change parameters obtained from microphones 612, 614 shown in FIG. 6C. Each of the first and second ear cavity acoustic change parameters includes a waveform representative of the change in shape and rigidity of the respective sealed ear cavities 606, 608 as a function of time in response to an acoustic input introduced into the sealed ear cavities (this waveform may be referred to as an acoustic change waveform). Each of these waveforms relates to a blood pulse waveform (as a function of time) associated with the respective external carotid arteries 136, 142 since a change in shape and rigidity occurs each time a blood pulse is present at the external carotid arteries 136, 142. The slight difference in the arrival of each given blood pulse between the right ear 110 and the left ear 118 (Δ PAT) is derived from the two blood pulse waveforms. The Δ PAT relates to PWV, and PWV relates to CABP. In other words, the acoustic change waveforms have a certain functional relationship with Δ PAT, and Δ PAT enjoys a specific functional relationship with CABP: acoustic change waveforms=f(Δ PAT) and Δ PAT=f(CABP).

In one embodiment, the relationship or correlation between the acoustic change waveforms and Δ PAT can be empirically derived. As an example, the right and left earphones 102, 104 can be configured to include the pressure transducers 602, 604 (shown in FIG. 6A) and the microphones 612, 614 (shown in FIG. 6C) (along with the speakers 611, 613 that would be typically included in earphones for normal audio operations). For each subject in this study, pressure change measurements are obtained from the pressure transducers 602, 604 (Δ PAT) simultaneously with acoustic change measurements that are obtained from the microphones 612, 614 (acoustic change waveforms). From these data points from all the study subjects, a relationship between Δ PAT and the acoustic change waveforms can be inferred. Once this relationship is known, the same known algorithmic methods or empirically-derived relationship between Δ PAT and CABP discussed above with respect to sub-block 312a can be used to convert the first and second ear cavity acoustic change parameters obtained from microphones 612, 614 to CABP. As discussed above with respect to sub-block 312a, empirically-derived relationships may also be used for calibration purposes (the calibration in this case involving use of the microphones 612, 614 and an acoustic input instead of the pressure transducers 602, 604).

At sub-block 312d, the physiological measurement module 410 is configured to determine a carotid/central blood pressure measurement based on the first ear cavity acoustic change parameter obtained from one of the microphones 612, 614 as shown in FIG. 6D. The first ear cavity acoustic change parameter includes a waveform representative of the change in shape and rigidity of the sealed ear cavity 606 as a function of time in response to an acoustic input introduced into the sealed ear cavity 606 (this waveform may be referred to as an acoustic change waveform). This waveform relates to a blood pulse waveform (as a function of time) associated with the external carotid artery 136 since a change in shape and rigidity occurs each time a blood pulse is present at the external carotid artery 136. This blood pulse waveform, in turn, is representative of the external carotid artery blood pressure (also referred to as the carotid blood pressure). The conversion to the carotid blood pressure can be performed using known conversion algorithmic methods, such as Principle components regression. Assuming the carotid blood pressure is sufficiently the same as CABP, this sensor configuration uses a single tonometer to provide the central and carotid blood pressure measurements.

Next at sub-block 312e, the physiological measurement module 410 is configured to determine a central aortic blood pressure measurement based on the first ear cavity pressure change parameter obtained from the pressure transducer 602, the second electrical parameter obtained from the first electrode 622, and the third electrical parameter obtained from the second electrode 624, all shown in FIG. 6E. The first and second electrical parameters together form a waveform representative of the depolarization times of the heart 126, in which the heart 126 ejects or pumps out a blood bolus with each depolarization. The peaks of this waveform are also referred to as ECG spikes. The first ear cavity pressure change parameter includes a waveform of this blood bolus or pulse (subsequently) arriving at the external carotid artery 136 near the right ear 110 as a function of time. The difference in a given depolarization time and the corresponding pulse arrival time at the external carotid artery 136 is a Δ PAT. As discussed above, there is a certain functional relationship between Δ PAT and PWV and another functional relationship between PWV and CABP: Δ PAT=f(PWV) and PWV=f(CABP).

In one embodiment, the translation or conversion of measured Δ PAT to CABP can be performed using known algorithmic methods that specify the quantitative relationship or correlation between PWV and CABP. As an example, reference is made to http://en.wikipedia.org/wiki/Pulse_wave_velocity that provides example algorithmic methods for the functional relationship between PWV and CABP. The article includes the following equation showing the relationship between PWV and P (arterial blood pressure CABP):

$$PWV = \sqrt{\frac{dP \cdot V}{ρ \cdot dV}} ,$$

where ρ is the density of blood and V is the blood volume. The article also provides an alternative expression of PWV as a function of P (arterial blood pressure CABP):

PWV=Pᵢ/(vᵢ·ρ)=Z_{c}/ρ,

where v is the blood flow velocity (in the absence of wave reflection) and P is the density of blood. The PWV may be determined by measuring the distance along the arterial tree between the pressure transducer 602 and the heart 126 (e.g., using a tape measure). Then this distance is divided by the measured Δ PAT resulting in the PWV. With PWV known, a conversion algorithmic method referenced above can be applied to convert PWV to CABP.

In another embodiment, the relationship or correlation between Δ PAT and CABP for this three-sensor configuration can be empirically derived. Similar to the empirical derivation discussion above with respect to sub-block 312a, a human study involving a relatively small number of subjects can be conducted. For each subject, three simultaneous measurements are obtained: (1) the Δ PAT measurement using the three sensor configuration shown in FIG. 6E, (2) brachial BP measurement using a brachial cuff, and (3) direct CABP measurement using a catheter with a pressure sensor deployed within the aortic arch 128. Based on these simultaneous measurements from all the subjects, relationships among Δ PAT, brachial BP, and actual CABP with each other can be derived. With these empirically-derived relationships, the measured Δ PAT can be converted into a CABP measurement. The empirically-derived relationships can also be used in the calibration process of block 302 to determine a scaling factor applicable to the particular individual using this three-sensor configuration to obtain a CABP measurement.

At sub-block 312f, the physiological measurement module 410 is configured to determine a central aortic blood pressure measurement based on the first ear cavity acoustic change parameter obtained from the microphone 612, the second electrical parameter obtained from the first electrode 622, and the third electrical parameter obtained from the second electrode 624, all shown in FIG. 6F. This particular three-sensor configuration also provides Δ PAT measurements, albeit using an acoustic input to the sealed ear cavity 606 (e.g., operating in active mode) as opposed to the passive mode of FIG. 6E/sub-block 306e. Accordingly, the discussion above for sub-block 312e regarding conversion of Δ PAT to CABP is also applicable for sub-block 312f.

Next at essub-block 312g, the physiological measurement module 410 is configured to determine an ECG measurement based on the first electrical parameter from the first electrode 632 and the second electrical parameter from the second electrode 634, as shown in FIG. 6G. In one embodiment, the ECG measurements include Lead 1 ECG signal measurements. The detected Lead 1 ECG signals may undergo little or no processing/conversion to form the final ECG measurements. In another embodiment, the Lead 1 ECG signals may be converted into a heart rate measurement (also referred to as a pulse measurement) using known algorithmic methods. An example algorithmic method is discussed at http://courses.kcumb.edu/physio/ecg%20primer/normecgcalcs.htm#The%20R-R%20interval, which discusses identifying a particular point on consecutive signals of the ECG waveform and using the known time difference between such particular points on the consecutive signals to obtain the number of heart beats per unit of time.

At sub-block 312h, the physiological measurement module 410 is configured to determine a core body temperature measurement based on the first temperature parameter obtained from the temperature sensor 642, as shown in FIG. 6H. In one embodiment, the first temperature parameter undergoes little or no processing/conversion to form the core body temperature measurement. As an example, the core body temperature may merely be a conversion of the first temperature parameter in accordance with a conversion table or equation.

At sub-block 312i, the physiological measurement module 410 is configured to determine a skin surface temperature measurement or stress/relaxation level indication based on the first temperature parameter obtained from the temperature sensor 652, as shown in FIG. 6I. In one embodiment, the first temperature parameter undergoes little or no processing/conversion to output a skin surface temperature measurement. As an example, the skin temperature may merely be a conversion of the first temperature parameter in accordance with a conversion table or equation. In another embodiment, a known or empirically-derived correlation between the skin surface temperature and stress level can be used to provide a stress/relaxation level indication based on the first temperature parameter. An example of a suitable conversion algorithmic method includes building a model of skin temperature in a cohort while invoking a fight or flight response.

At sub-block 312j, the physiological measurement module 410 is configured to determine a galvanic skin response measurement or stress/relaxation level indication based on the first and second impedance parameters obtained from the first and second electrodes 662, 663, as shown in FIG. 6J. The first and second impedance parameters include a measure of the moisture level of the user's skin at the contact areas. The skin moisture level relates to galvanic skin response, and galvanic skin response is indicative of stress/relaxation level. Known or empirically-derived correlations between the skin moisture level, galvanic skin response, and stress/relaxation levels can be used to translate the first and second impedance parameters into the galvanic skin response measurement and/or stress/relaxation level indication. An example of a suitable conversion algorithmic method includes building a model of galvanic skin response in a cohort while invoking a fight or flight response.

At sub-block 312k, the physiological measurement module 410 is configured to determine a body fat content measurement and/or a body water content measurement based on the first and second impedance parameters obtained from the first and second electrodes 672, 674, as shown in FIG. 6K. Use of body impedance information to generate physiological measurement includes bioelectrical impedance analysis (BIA) measurements. For at least the body fat content measurement, the first and second impedance parameters may be converted to corresponding body fat content using known algorithmic methods, such algorithmic method taking into account the user's weight, height, gender, and/or age (previously provided by the user 108 at calibration block 302). In other embodiments, known algorithmic methods may be used for each of body fat content and body water content determination without calibration information. Examples of suitable algorithmic methods for body fat content determination are provided in Ursula G. Kyle et al., “Bioelectrical impedance analysis—part I: review of principles and methods,” Clinical Nutrition, Vol. 23 (5): 1226-1243 (2004), and G. Bedogni et al., “Accuracy of an eight-point tactile-electrode impedance method in the assessment of total body water,” European Journal of Clinical Nutrition, Vol. 56, 1143-1148 (2002) (available at http://www.nature.com/ejcn/journal/v56/n11/full/1601466a.html) for body water content determination. Tables 2 and 3 of the Kyle article provide a survey of equations reported in other articles for calculating the body fat as a function of the subject's measured resistance (which is quantitatively related to impedance), height, weight, age, gender, and/or other variables. Since these equations provide an estimation of the body fat, the amount of error inherent in each of the equations is also provided in the tables. For body water content determination, the Bedogni article provides tables and plots to empirically translate measured resistance for a certain body part (e.g., trunk, right arm, left arm, right leg, left leg) to a resistance value for the whole body and from that to the body water content value (referred to as total body water (TBW) in the article). See also Nawarycz, T, et al., “Electroimpedance measurements of body composition employing the method of double sampling,” Engineering in Medicine and Biology Society, Bridging Disciplines for Biomedicine, Proceedings of the 18^{th} Annual International Conference of the IEEE, Vol. 5, 1932-1933 (Oct. 31-Nov. 3, 1996), available at http://ieeexplore.ieee.org/stamp/stamp.jsp?tp=&arnumber=646326&isnumber=14105.

With the determination of physiological measurement(s) completed in block 312, the information display module 404 is configured to facilitate display of one or more user interface screens including such physiological measurement(s) on the touch sensor panel 150 (block 314). Associated information about the presented physiological measurement(s) may also be provided on the touch sensor panel 150 to aid the user 108 in understanding the measurements. For blood pressure measurements, for example, different range values and what each range means may be provided and for those range values indicative of health issues, recommendations may be given to see a doctor right away or the like.

Last, at block 316, the calculated physiological measurement(s) along with related information (e.g., time and date stamp, user identifier, etc.) can be saved in the portable device 106 and/or transmitted to another device. A post-calculation module 412 is configured to facilitate saving the data to a memory included in the portable device 106. The post-calculation module 412 is also configured to facilitate transmission of the physiological measurement(s) (and their associated information) over a network, such as over a cellular network or a WiFi network, to a remote device (e.g., another portable device, server, database, etc.). By saving and/or communicating one or more physiological measurements over time, such information may illuminate trends for useful health assessment.

It is understood that one or more of blocks 302-316 may be performed in a different sequence than shown in FIG. 3A. For example, block 316 may be performed prior to or simultaneously with block 314. Sub-blocks 312a-k of FIG. 3C may be performed in any sequential order, or simultaneously with each other depending on, for example, when a set of physiological parameters are received by the portable device 106 and/or the processing capacity of the portable device 106.

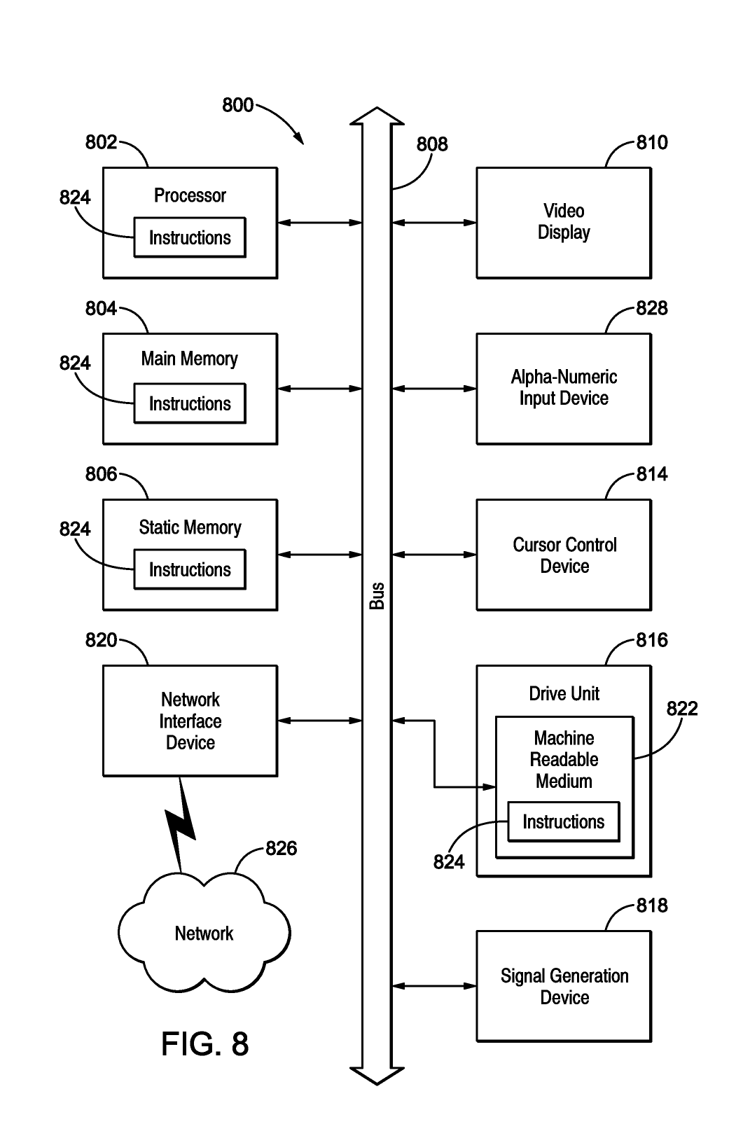

FIG. 8 depicts a block diagram representation of an example architecture for the controller assembly 152. Although not required, many configurations for the controller assembly 152 can include one or more microprocessors which will operate pursuant to one or more sets of instructions for causing the machine to perform any one or more of the methodologies discussed herein.

The example controller assembly 800 includes a processor 802 (e.g., a central processing unit (CPU), a graphics processing unit (GPU) or both), a main memory 804 and a static memory 806, which communicate with each other via a bus 808. The controller assembly 800 may further include a video display unit 810 (e.g., a liquid crystal display (LCD) or a cathode ray tube (CRT)). The controller assembly 800 may also include an alphanumeric input device 812 (e.g., a keyboard, mechanical or virtual), a cursor control device 814 (e.g., a mouse or track pad), a disk drive unit 816, a signal generation device 818 (e.g., a speaker), and a network interface device 820.

The disk drive unit 816 includes a machine-readable storage medium 822 on which is stored one or more sets of executable instructions (e.g., apps) embodying any one or more of the methodologies or functions described herein. In place of the disk drive unit, a solid-state storage device, such as those including flash memory may be utilized. The executable instructions may also reside, completely or at least partially, within the main memory 804 and/or within the processor 802 during execution thereof by the controller assembly 800; the main memory 804 and the processor 802 also constituting machine-readable storage media. Alternatively, the instructions may be only temporarily stored on a machine-readable medium within controller 800, and until such time may be received over a network 826 via the network interface device 820.

While the machine-readable medium 822 is shown in an example embodiment to be a single medium, the term “machine-readable medium” as used herein should be taken to include a single medium or multiple media (e.g., a centralized or distributed database, and/or associated caches and servers) that store the one or more sets of instructions. The term “machine-readable medium” or “computer-readable medium” shall be taken to include any tangible non-transitory medium (which is intended to include all forms of memory, volatile and non-volatile) which is capable of storing or encoding a sequence of instructions for execution by the machine.

Many additional modifications and variations may be made in the techniques and structures described and illustrated herein without departing from the spirit and the scope of the present invention. Accordingly, the present invention should be clearly understood to be limited only by the scope of the claims and equivalents thereof.

1. A portable system for obtaining one or more physiological measurements, comprising:
a first sensor assembly configured to sealingly engage a first ear of a user to form a first sealed ear cavity within a first ear canal, the first sensor assembly including a first sensor configured to detect a first physiological parameter associated with the first sealed ear cavity and to generate a signal representative of the physiological parameter, wherein the first sensor comprises a pressure sensor configured to detect changes in pressure within the first sealed ear cavity that is indicative of a blood pulse pressure; and
a processor in communication with the first sensor assembly, the processor configured to receive the first physiological parameter signal from the first sensor assembly and to determine a first physiological measurement based on the received first physiological parameter.

2. The portable system of claim 1, further comprising a touch-sensitive display in communication with the processor, the touch-sensitive display and the processor being included in a hand-held device.

3. The portable system of claim 2, wherein the processor is configured to display instructions on the touch sensitive display for a user to use the system to generate the first physiological measurement.

4. The portable system of claim 2, wherein the portable device comprises one of a smart phone, a tablet, an audio device, a video device, a laptop, and a computing device.

5. The portable system of claim 1, wherein the first physiological parameter comprises a blood pulse waveform associated with at least a portion of an external carotid artery located proximate to the first sealed ear cavity, and wherein the first physiological measurement comprises a blood pressure measurement.

6. The portable system of claim 1, wherein the first sensor comprises a temperature sensor and wherein the first physiological measurement comprises a body temperature measurement.

7. The portable system of claim 1, further comprising:
a second sensor assembly configured to sealingly engage the second ear of a user to form a second sealed ear cavity within a second ear canal, the second sensor assembly having a second sensor configured to detect a second physiological parameter associated with the second ear canal;
wherein the processor is in communication with the second sensor assembly, and wherein the processor is further configured to receive the second physiological parameter from the second earphone and to determine the first physiological measurement based on the first and the second physiological parameters.

8. The portable system of claim 7, wherein each of the first and the second sensors comprises a pressure sensor, and wherein the first physiological measurement comprises a central blood pressure measurement.

9. The portable system of claim 7, wherein each of the first and the second sensors comprises an electrode, and the first physiological measurement comprises an electrocardiogram (ECG) measurement.

10. The portable system of claim 7, wherein each of the first and the second sensors comprises an electrode, and the first physiological measurement comprises a body fat content measurement or a body water content measurement.

11. The portable system of claim 1, further comprising:
a second sensor configured to obtain a second physiological parameter; and
a third sensor configured to obtain a third physiological parameter, wherein the first sensor comprises a pressure sensor, and wherein the processor is configured to determine an electrocardiogram (ECG) spike from the second and the third physiological parameters, and to determine the first physiological measurement comprising a blood pressure measurement based on the first, the second, and the third physiological parameters.

12. The portable system of claim 1, wherein the first sensor assembly comprises a second sensor located at a different location from the first sensor, the second sensor configured to obtain a second physiological parameter, and wherein the processor is configured to determine the first physiological measurement comprising a galvanic skin response measurement based on the first and the second physiological parameters.

13. The portable system of claim 1, further comprising:
a second sensor assembly including a second sensor, the second sensor assembly configured to seal a second ear canal to form a second sealed ear cavity within the second ear canal and the second sensor configured to detect a second physiological parameter associated with the second ear canal,
wherein the first sensor assembly includes a third sensor configured to detect a third physiological parameter associated with the first ear canal, each of the second and the third sensors comprising a conductive contact area, and wherein the processor is configured to determine a second physiological measurement based on the second and the third physiological parameters.

14. The system of claim 1, further comprising a transmitter in communication with the processor, the transmitter configured to transmit the first physiological measurement to a remote device.

15. A method for obtaining one or more physiological measurements, the method comprising:
receiving, from a first earphone sealingly engaging a first ear of a user, a first signal representative of a first physiological parameter associated with a first ear canal of a user, the first physiological parameter measured by a first sensor comprising a pressure sensor included in the first earphone for detecting changes in pressure within the first ear canal that is indicative of a blood pulse pressure, the first earphone configured to seal the first ear canal to form a first sealed ear cavity within the first ear canal, wherein the earphone is configured to communicate audio information to the user's ear; and
generating a first physiological measurement based at least in part on the first physiological parameter using a physiological measurement module associated with the first earphone.

16. The method of claim 15, further comprising receiving, from a second earphone sealingly engaging a second ear of a user, a second signal representative of a second physiological parameter associated with a second ear canal of the user, the second physiological parameter measured by a second sensor included in the second earphone, the second earphone configured to seal the second ear canal to form a second ear cavity within the second ear canal, wherein the generating of the first physiological measurement comprises generating the first physiological measurement using the first and the second physiological parameters.

17. The method of claim 16, wherein each of the first and the second sensors comprises a pressure transducer, and wherein the first physiological measurement comprises a blood pressure measurement.

18. The method of claim 16, wherein each of the first and the second sensors comprises an electrode, and wherein the first physiological measurement comprises an electrocardiogram (ECG) measurement.

19. The method of claim 15, wherein the first sensor comprises a temperature sensor and the first physiological measurement comprises a body temperature measurement.

20. The method of claim 15, further comprising:
receiving a second physiological parameter associated with the first ear canal of the user, the second physiological parameter measured by a second sensor included in the first earphone, wherein the generating of the first physiological measurement comprises generating the first physiological measurement using the first and the second physiological parameters.

21. The method of claim 15, further comprising transmitting the first physiological measurement to a remote device.

22. The method of claim 15, wherein the generating of the first physiological measurement is performed by a portable device in communication with the first earphone.

23. A portable apparatus for obtaining one or more physiological measurements, comprising:
a device assembly, the device comprising at least one processor;
a first earphone including a first sensor comprising a pressure sensor, the first earphone in communication with the device assembly, the first earphone configured to seal a first ear canal of a user to form a first sealed ear cavity within the first ear canal and the first sensor configured to detect changes in pressure within the first sealed ear cavity that is indicative of a blood pulse pressure and corresponds to a first physiological parameter associated with the first ear canal;
a second earphone including a second sensor, the second earphone in communication with the device assembly, the second earphone configured to seal a second ear canal of the user to form a second sealed ear cavity within the second ear canal and the second sensor configured to detect a second physiological parameter associated with the second ear canal; and
a display in communication with the processor, the touch-sensitive display configured to determine and display a physiological measurement corresponding to at least one of the first and the second detected physiological parameters.

24. The portable apparatus of claim 23, wherein the physiological measurement comprises a blood pressure measurement, an electrocardiogram (ECG) measurement, a heart rate measurement, a bioelectrical impedance analysis (BIA) measurement, a body temperature measurement, a galvanic skin response measurement, a stress level indication measurement, a body water content measurement, or a body fat content measurement.
